(19) United States
(12) Patent Application Publication
(10) Pub. No.: US 2026/0273050 A1
(43) Pub. Date: Sep. 17, 2026

(54) COMBINATION TREATMENTS COMPRISING ANTICANCER AGENTS AND AN ANTI-TGFßR2/PD-1 BISPECIFIC ANTIBODY

(71) Applicant: Incyte Corporation, Wilmington, DE (US)

(72) Inventors: Thomas Karasic, Wilmington, DE (US); Chiara Greggio, Morges (CH)

(21) Appl. No.: 19/565,712

(22) Filed: Mar. 13, 2026

Related U.S. Application Data
(60) Provisional application No. 63/772,224, filed on Mar. 14, 2025.
(60) Provisional application No. 63/969,023, filed on Jan. 27, 2026.

(51) Int. Cl. A61K 39/395; A61K 31/282; A61K 31/4745; A61K 31/513; A61K 31/519; A61K 39/00; A61P 35/00

(52) U.S. Cl. CPC A61K 39/3955; A61K 31/282; A61K 31/4745; A61K 31/513; A61K 31/519; A61P 35/00; A61K 2039/507; A61K 2039/54; A61K 2039/545

(57) ABSTRACT

The present disclosure describes a combination treatment comprising (1) an anticancer therapy and (2) a bispecific antibody targeting human programmed death-1 (PD-1) and human transforming growth factor β receptor 2 (TGFβR2) for use in the treatment of disorder such as non-small cell lung cancer (NSCLC), squamous cell carcinoma of the head and neck (SCCHN), cervical squamous cell carcinoma and endocervical adenocarcinoma (CESC), ovarian cancer, breast cancer, bladder cancer, renal cell carcinoma, melanoma, gastric adenocarcinoma, esophageal cancer, gastroesophageal adenocarcinoma, malignant pleural mesothelioma, pancreatic adenocarcinoma, and colorectal cancer (CRC).

### CROSS-REFERENCE TO RELATED APPLICATIONS

This application claims the benefit of priority to U.S. Provisional Patent Application No. 63/772,224, filed on Mar. 14, 2025, and U.S. Provisional Patent Application No. 63/969,023, filed on Jan. 27, 2026, the entire contents of which are hereby incorporated by reference in their entirety.

### SEQUENCE LISTING

The instant application contains a Sequence Listing which has been submitted electronically in XML format and is hereby incorporated by reference in its entirety. Said XML copy, created on Mar. 6, 2026, is named 20443-0889001_SL.xml and is 14,632 bytes in size.

### TECHNICAL FIELD

The present invention is directed to treating cancer by administering to a subject a combination treatment comprising (1) an anti-cancer therapy and (2) a bispecific antibody that binds to human TGFβR2 and human PD-1.

### BACKGROUND

Cancer is among the leading causes of death worldwide. Many patients are diagnosed with advanced disease, have no response to treatment, or have a response to treatment that is followed by disease progression. Thus, there is a need for therapies targeting cancer.

### SUMMARY

Provided herein are methods of treating a disorder in a human subject in need thereof, wherein the disorder is selected from the group consisting of a non-small cell lung cancer (NSCLC), a squamous cell carcinoma of the head and neck (SCCHN), a cervical squamous cell carcinoma and endocervical adenocarcinoma (CESC), an ovarian cancer, a breast cancer, a bladder cancer, a renal cell carcinoma, a melanoma, a gastric adenocarcinoma, an esophageal cancer, a gastroesophageal adenocarcinoma, a malignant pleural mesothelioma, a pancreatic adenocarcinoma, and a colorectal cancer (CRC), wherein the method comprises administering to the human subject a combination treatment comprising (1) a therapeutically effective amount of an anticancer therapy and (2) a therapeutically effective amount of a bispecific antibody that binds to human programmed death-1 (PD-1) and human transforming growth factor β receptor 2 (TGFβR2), wherein the anticancer therapy comprises one or more of bevacizumab, FOLFIRI, FOLFOX, or cetuximab, wherein the bispecific antibody comprises: an anti-human PD-1 binding domain comprising a PD-1 heavy chain variable region and a PD-1 light chain variable region, wherein the PD-1 heavy chain variable region comprises heavy chain CDR1 (HCDR1) comprising the amino acid sequence RFALH (SEQ ID NO: 1), heavy chain CDR2 (HCDR2) comprising the amino acid sequence WIDPNTGTPTFAQGVTG (SEQ ID NO:2), and heavy chain CDR3 (HCDR3) comprising the amino acid sequence SLGYCDSDICYPNWIFDN (SEQ ID NO:3), and wherein the PD-1 light chain variable region comprises light chain CDR1 (LCDR1) comprising the amino acid sequence QSISSY (SEQ ID NO:11), light chain CDR2 (LCDR2) comprising the amino acid sequence AAS, and light chain CDR3 (LCDR3) comprising the amino acid sequence QQSYSTPPT (SEQ ID NO:13); and an anti-human TGFβR2 binding domain comprising a TGFβR2 heavy chain variable region and a TGFβR2 light chain variable region, wherein the TGFβR2 heavy chain variable region comprises HCDR1 comprising the amino acid sequence IYAMT (SEQ ID NO:6), HCDR2 comprising the amino acid sequence VISGSGGTTYYADSVKG (SEQ ID NO:7), and HCDR3 comprising the amino acid sequence RGQYRDIVGATDY (SEQ ID NO:8), and wherein the TGFβR2 light chain variable region comprises LCDR1 comprising the amino acid sequence QSISSY (SEQ ID NO: 11), LCDR2 comprising the amino acid sequence AAS, and LCDR3 comprising the amino acid sequence QQSYSTPPT (SEQ ID NO:13).

In some embodiments, the PD-1 heavy chain variable region comprises the amino acid sequence QVQLVQSGSELKKPGASVKVSCKASGYTFTRFALHWVRQAPGQGLEWMGWIDPNT GTPTFAQGVTGRFVFSLDTSVTTAYLQISSLKAEDTAVYYCARSLGYCDSDICYPNWI FDNWGQGTLVTVSS (SEQ ID NO:4) and the TGFβR2 heavy chain variable region comprises the amino acid sequence

(SEQ ID NO: 9)
EVQLVESGGGLVQPGGSLRLSCAASGFTFDIYAMTWVRQAPGKGLEWVS
VISGSGGTTYYADSVKGRFTISRDNSKNTLYLQMNSLRAEDTAVYYCAR
RGQYRDIVGATDYWGQGTLVTVSS.

In some embodiments, the PD-1 light chain variable region and the TGFβR2 light chain variable region each comprise the amino acid sequence

(SEQ ID NO: 14)
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPPTF
GQGTKVEIK.

In some embodiments, the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO:5, the PD-1 light chain light chain comprises the amino acid sequence set forth in SEQ ID NO:15, the TGFβR2 heavy chain comprises the amino acid sequence set forth in SEQ ID NO: 10, and the TGFβR2 light chain light chain comprises the amino acid sequence set forth in SEQ ID NO: 15.

In some embodiments, the anticancer therapy comprises bevacizumab.

In some embodiments, the anticancer therapy comprises bevacizumab and FOLFIRI.

In some embodiments, the anticancer therapy comprises bevacizumab and FOLFOX.

In some embodiments, the anticancer therapy comprises cetuximab.

In some embodiments, the human subject has a NSCLC. In some instances, the NSCLC has squamous or nonsquamous histology. In some instances, the NSCLC is advanced or metastatic.

In some embodiments, the human subject has a SCCHN. In some instances, the SCCHN comprises a primary squamous tumor of the oral cavity, oropharynx, hypopharynx, or larynx. In some instances, the SCCHN is advanced or metastatic.

In some embodiments, the human subject has a CESC. In some instances, the CESC is advanced or metastatic.

In some embodiments, the human subject has human papilloma virus (HPV)-positive SCCHN or HPV-positive CESC.

In some embodiments, the human subject has an ovarian cancer. In some instances, the ovarian cancer comprises ovarian epithelial carcinoma, fallopian tube carcinoma, primary peritoneal carcinoma, or carcinosarcoma. In some instances, the ovarian cancer is advanced or metastatic.

In some embodiments, the human subject has a breast cancer. In some instances, the breast cancer comprises a triple negative breast cancer. In some instances, the triple negative breast cancer comprises HER2-negative, ER-negative, and PgR-negative breast cancer. In some instances, the breast cancer is advanced or metastatic.

In some embodiments, the human subject has a bladder cancer. In some instances, the bladder cancer comprises urothelial carcinoma. In some instances, the bladder cancer is advanced or metastatic.

In some embodiments, the human subject has a renal cell carcinoma. In some instances, the renal cell carcinoma is advanced or metastatic.

In some embodiments, the human subject has a melanoma. In some instances, the melanoma is cutaneous malignant melanoma. In some instances, the melanoma is advanced or metastatic.

In some embodiments, the human subject has a gastric adenocarcinoma. In some instances, the gastric adenocarcinoma is advanced or metastatic.

In some embodiments, the human subject has an esophageal cancer. In some instances, the esophageal cancer is advanced or metastatic.

In some embodiments, the human subject has a gastroesophageal adenocarcinoma. In some instances, the gastroesophageal adenocarcinoma is advanced or metastatic.

In some embodiments, the human subject has a malignant pleural mesothelioma. In some instances, the malignant pleural mesothelioma is advanced or metastatic.

In some embodiments, the human subject has a pancreatic adenocarcinoma. In some instances, the pancreatic adenocarcinoma is advanced or metastatic.

In some embodiments, the human subject has a CRC. In some instances, the CRC comprises microsatellite stable colorectal cancer (MSS-CRC). In some instances, the CRC comprises deficient mismatch repair (dMMR)/high microsatellite instability (MSI-H) colorectal cancer (dMMR/MSI-H CRC). In some instances, the CRC is advanced or metastatic.

In some embodiments, the human subject has experienced disease progression after prior treatment. In some instances, the prior treatment comprises anti-PD-(L)1 therapy and/or anti-CTLA4 therapy.

In some embodiments, the disorder is nonamenable to curative treatments or procedures.

In some embodiments, the bispecific antibody is administered intravenously.

In some embodiments, the bispecific antibody is administered at a dose of about 100 mg, about 300 mg, about 900 mg, about 1500 mg, or about 2000 mg.

In some embodiments, the bispecific antibody is administered intravenously at a dose of about 100 mg, about 300 mg, about 900 mg, about 1500 mg, or about 2000 mg.

In some embodiments, the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO:5, the PD-1 light chain light chain comprises the amino acid sequence set forth in SEQ ID NO:15, the TGFβR2 heavy chain comprises the amino acid sequence set forth in SEQ ID NO: 10, and the TGFβR2 light chain light chain comprises the amino acid sequence set forth in SEQ ID NO: 15, and wherein the bispecific antibody is administered intravenously at a dose of about 100 mg, about 300 mg, about 900 mg, about 1500 mg, or about 2000 mg.

In some embodiments, the bispecific antibody is administered once every two weeks.

In some embodiments, the bispecific antibody is administered intravenously once every two weeks at a dose of about 100 mg, about 300 mg, about 900 mg, about 1500 mg, or about 2000 mg.

In some embodiments, the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO:5, the PD-1 light chain light chain comprises the amino acid sequence set forth in SEQ ID NO:15, the TGFβR2 heavy chain comprises the amino acid sequence set forth in SEQ ID NO:10, and the TGFβR2 light chain light chain comprises the amino acid sequence set forth in SEQ ID NO: 15, and wherein the bispecific antibody is administered intravenously once every two weeks at a dose of about 100 mg, about 300 mg, about 900 mg, about 1500 mg, or about 2000 mg.

In some embodiments, the bispecific antibody is administered once every four weeks. In some instances, the bispecific antibody is administered intravenously once every four weeks at a dose of about 100 mg, about 300 mg, about 900 mg, about 1500 mg, or about 2000 mg.

In some embodiments, the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO:5, the PD-1 light chain light chain comprises the amino acid sequence set forth in SEQ ID NO:15, the TGFβR2 heavy chain comprises the amino acid sequence set forth in SEQ ID NO: 10, and the TGFβR2 light chain light chain comprises the amino acid sequence set forth in SEQ ID NO: 15, and wherein the bispecific antibody is administered intravenously once every four weeks at a dose of about 100 mg, about 300 mg, about 900 mg, about 1500 mg, or about 2000 mg.

In some embodiments, the bispecific antibody is administered intravenously once every two weeks at a dose of about 900 mg.

In some embodiments, the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO:5, the PD-1 light chain light chain comprises the amino acid sequence set forth in SEQ ID NO: 15, the TGFβR2 heavy chain comprises the amino acid sequence set forth in SEQ ID NO:10, and the TGFβR2 light chain light chain comprises the amino acid sequence set forth in SEQ ID NO: 15, and wherein the bispecific antibody is administered intravenously once every two weeks at a dose of about 900 mg.

In some embodiments, the method is a method of treating metastatic microsatellite stable colorectal cancer in a human subject in need thereof, wherein the method comprises administering to the human subject a combination treatment comprising (1) a therapeutically effective amount of FOLFOX, (2) a therapeutically effective amount of bevacizumab, and (3) a therapeutically effective amount of a bispecific antibody that binds to human programmed death-1 (PD-1) and human transforming growth factor β receptor 2 (TGFβR2);
wherein the bispecific antibody comprises:
an anti-human PD-1 binding domain comprising a PD-1 heavy chain variable region and a PD-1 light chain variable region, wherein the PD-1 heavy chain variable region comprises heavy chain CDR1 (HCDR1) comprising the amino acid sequence RFALH (SEQ ID NO: 1), heavy chain CDR2 (HCDR2) comprising the amino acid sequence WIDPNTGTPTFAQGVTG (SEQ ID NO:2), and heavy chain CDR3 (HCDR3) comprising the amino acid sequence SLGYCDSDICYPNWIFDN (SEQ ID NO:3), and wherein the PD-1 light chain variable region comprises light chain CDR1 (LCDR1) comprising the amino acid sequence QSISSY (SEQ ID NO: 11), light chain CDR2 (LCDR2) comprising the amino acid sequence AAS, and light chain CDR3 (LCDR3) comprising the amino acid sequence QQSYSTPPT (SEQ ID NO:13); and
an anti-human TGFβR2 binding domain comprising a TGFβR2 heavy chain variable region and a TGFβR2 light chain variable region, wherein the TGFβR2 heavy chain variable region comprises HCDR1 comprising the amino acid sequence IYAMT (SEQ ID NO:6), HCDR2 comprising the amino acid sequence VISGSGGTTYYADSVKG (SEQ ID NO:7), and HCDR3 comprising the amino acid sequence RGQYRDIVGATDY (SEQ ID NO:8), and wherein the TGFβR2 light chain variable region comprises LCDR1 comprising the amino acid sequence QSISSY (SEQ ID NO:11), LCDR2 comprising the amino acid sequence AAS, and LCDR3 comprising the amino acid sequence QQSYSTPPT (SEQ ID NO:13).

In some embodiments, the PD-1 heavy chain variable region comprises the amino acid sequence QVQLVQSGSELKKPGASVKVSCKASGYTFTRFALHWVRQAPGQGLEWMGWIDPNT GTPTFAQGVTGRFVFSLDTSVTTAYLQISSLKAEDTAVYYCARSLGYCDSDICYPNWI FDNWGQGTLVTVSS (SEQ ID NO:4) and the TGFβR2 heavy chain variable region comprises the amino acid sequence

(SEQ ID NO: 9)
EVQLVESGGGLVQPGGSLRLSCAASGFTFDIYAMTWVRQAPGKGLEWVS
VISGSGGTTYYADSVKGRFTISRDNSKNTLYLQMNSLRAEDTAVYYCAR
RGQYRDIVGATDYWGQGTLVTVSS.

In some embodiments, the PD-1 light chain variable region and the TGFβR2 light chain variable region each comprise the amino acid sequence

(SEQ ID NO: 14)
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPPTF
GQGTKVEIK.

In some embodiments, the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO:5, the PD-1 light chain light chain comprises the amino acid sequence set forth in SEQ ID NO:15, the TGFβR2 heavy chain comprises the amino acid sequence set forth in SEQ ID NO:10, and the TGFβR2 light chain light chain comprises the amino acid sequence set forth in SEQ ID NO:15.

In some embodiments, the bispecific antibody is administered intravenously once every two weeks at a dose of about 900 mg.

In some embodiments, the method is a method of treating metastatic microsatellite stable colorectal cancer in a human subject in need thereof, wherein the method comprises administering to the human subject a combination treatment comprising (1) a therapeutically effective amount of FOLFOX, (2) a therapeutically effective amount of bevacizumab, and (3) a therapeutically effective amount of a bispecific antibody that binds to human programmed death-1 (PD-1) and human transforming growth factor β receptor 2 (TGFβR2), wherein the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO:5, the PD-1 light chain light chain comprises the amino acid sequence set forth in SEQ ID NO:15, the TGFβR2 heavy chain comprises the amino acid sequence set forth in SEQ ID NO:10, and the TGFβR2 light chain light chain comprises the amino acid sequence set forth in SEQ ID NO: 15, and wherein the bispecific antibody is administered intravenously once every two weeks at a dose of about 900 mg.

In some embodiments, provided is a method of treating metastatic microsatellite stable colorectal cancer in a human subject in need thereof, wherein the method comprises administering to the human subject a combination treatment comprising (1) a therapeutically effective amount of bevacizumab, and (2) a therapeutically effective amount of a bispecific antibody that binds to human programmed death-1 (PD-1) and human transforming growth factor β receptor 2 (TGFβR2); wherein the bispecific antibody comprises: an anti-human PD-1 binding domain comprising a PD-1 heavy chain variable region and a PD-1 light chain variable region, wherein the PD-1 heavy chain variable region comprises heavy chain CDR1 (HCDR1) comprising the amino acid sequence RFALH (SEQ ID NO:1), heavy chain CDR2 (HCDR2) comprising the amino acid sequence WIDPNTGTPTFAQGVTG (SEQ ID NO:2), and heavy chain CDR3 (HCDR3) comprising the amino acid sequence SLGYCDSDICYPNWIFDN (SEQ ID NO:3), and wherein the PD-1 light chain variable region comprises light chain CDR1 (LCDR1) comprising the amino acid sequence QSISSY (SEQ ID NO:11), light chain CDR2 (LCDR2) comprising the amino acid sequence AAS (SEQ ID NO:12), and light chain CDR3 (LCDR3) comprising the amino acid sequence QQSYSTPPT (SEQ ID NO:13); and an anti-human TGFβR2 binding domain comprising a TGFβR2 heavy chain variable region and a TGFβR2 light chain variable region, wherein the TGFβR2 heavy chain variable region comprises HCDR1 comprising the amino acid sequence IYAMT (SEQ ID NO:6), HCDR2 comprising the amino acid sequence VISGSGGTTYYADSVKG (SEQ ID NO:7), and HCDR3 comprising the amino acid sequence RGQYRDIVGATDY (SEQ ID NO:8), and wherein the TGFβR2 light chain variable region comprises LCDR1 comprising the amino acid sequence QSISSY (SEQ ID NO: 11), LCDR2 comprising the amino acid sequence AAS (SEQ ID NO: 12), and LCDR3 comprising the amino acid sequence QQSYSTPPT (SEQ ID NO:13).

In some embodiments, the PD-1 heavy chain variable region comprises the amino acid sequence QVQLVQSGSELKKPGASVKVSCKASGYTFTRFALHWVRQAPGQGLEWMGWIDPNT GTPTFAQGVTGRFVFSLDTSVTTAYLQISSLKAEDTAVYYCARSLGYCDSDICYPNWI FDNWGQGTLVTVSS (SEQ ID NO:4) and the TGFβR2 heavy chain variable region comprises the amino acid sequence

(SEQ ID NO: 9)
EVQLVESGGGLVQPGGSLRLSCAASGFTFDIYAMTWVRQAPGKGLEWVS
VISGSGGTTYYADSVKGRFTISRDNSKNTLYLQMNSLRAEDTAVYYCAR
RGQYRDIVGATDYWGQGTLVTVSS.

In some embodiments, the PD-1 light chain variable region and the TGFβR2 light chain variable region each comprise the amino acid sequence

(SEQ ID NO: 14)
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPPTF
GQGTKVEIK.

In some embodiments, the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO:5, the PD-1 light chain light chain comprises the amino acid sequence set forth in SEQ ID NO: 15, the TGFβR2 heavy chain comprises the amino acid sequence set forth in SEQ ID NO: 10, and the TGFβR2 light chain light chain comprises the amino acid sequence set forth in SEQ ID NO:15.

In some embodiments, the bispecific antibody is administered intravenously once every two weeks at a dose of about 900 mg.

In some embodiments, the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO:5, the PD-1 light chain light chain comprises the amino acid sequence set forth in SEQ ID NO: 15, the TGFβR2 heavy chain comprises the amino acid sequence set forth in SEQ ID NO:10, and the TGFβR2 light chain light chain comprises the amino acid sequence set forth in SEQ ID NO: 15, and wherein the bispecific antibody is administered intravenously once every two weeks at a dose of about 900 mg.

In some embodiments, the cancer has progressed after treatment with a standard systemic therapy.

In some embodiments, the subject previously received fluoropyrimidine-, oxaliplatin-, and irinotecan-based chemotherapy.

In some embodiments, the subject previously received anti-VEGF and/or anti-EGFR therapy.

In some embodiments, the subject previously received trifluridine and tipiracil, regorafenib, and/or fruquintinib therapy.

In some embodiments, the method achieves an objective response rate of at least 30% in a plurality of treated subjects as assessed by RECIST v1.1.

### DETAILED DESCRIPTION

The present disclosure provides, in part, methods of treating cancer by administering a bispecific antibody targeting TGFβR2 and PD-1 in combination with one or more of the following therapies: 1) bevacizumab (an anti-VEGF antibody), 2) FOLFIRI (a combination of folinic acid (leucovorin), 5-fluorouracil (5-FU), and irinotecan), 3) FOLFOX (a combination of leucovorin, 5-FU, and oxaliplatin), and 4) cetuximab (an anti-EGFR antibody).

### Anti-TGFβR2/PD-1 Bispecific Antibodies

Transforming growth factor β is a multifunctional cytokine that acts in a cell- and context-dependent manner as a tumor promoter or tumor suppressor. Within healthy cells, TGFβ halts the cell cycle at the growth 1 phase, resulting in reduced proliferation and induction of differentiation while it may also promote apoptosis. In cancer cells, the TGFβ signaling pathway is deregulated or altered, and TGFβ no longer has the ability to control cellular proliferation. In mammals, there are 3 highly homologous TGFβ isoforms: TGFβ1, TGFβ2, and TGFβ3. The most prevalent, TGFβ1, is expressed in the majority of human cancer types. In addition, TGFβ1 expression has been the isoform most closely correlated with TGFβ signaling activation compared with isoforms TGFβ2 and TGFβ3.

TGFβ is synthesized as an inactive precursor that must be activated to allow for engagement of a tetrameric receptor complex composed of TGFβR1 and TGFβR2. Transforming growth factor β receptor 2 is a membrane-bound serine/threonine kinase that binds TGFβ1 and TGFβ3 with relative high affinity and TGFβ2 with lower affinity. Formation of a heterodimeric complex with TGFβR1 is required for signaling transduction following binding of the TGFβ ligand. Activated TGFβR2 phosphorylates multiple serine and threonine residues in the intracellular domain of TGFβR1, leading to its activation. Activated TGFβR1 then mediates activation of a downstream signaling pathway involving SMAD proteins that regulate target gene expression.

Programmed Cell Death 1 protein (PD-1, also known as CD279) is a cell surface receptor expressed on CD4+ and CD8+ T cells, B cells, NK cells, and myeloid-derived cells. PD-1 binds to two distinct ligands, PD-L1 and PD-L2, which differ in their expression patterns. PD-L1 (also known as B7-H1 or CD274) is expressed on hematopoietic cells such as T cells, B cells, dendritic cells, and macrophages, as well as an array of peripheral tissues, and PD-L1 expression levels are inducible by interferons. In contrast, PD-L2 (also known as B7-DC or CD273) expression is generally restricted to professional APCs and inducible by IL-4 and IL-10, depending on the lineage subset of the APC.

Binding of PD-1 to either PD-L1 or PD-L2 on T cells or B cells results in clustering with TCRs or BCRs and transient association with SH2 domain-containing tyrosine phosphatase 2. In turn, this induces a negative signal by dephosphorylating effector molecules that drive positive TCR and BCR signaling. This includes CD28-mediated activation of PI3K and subsequently Akt, glucose metabolism, and the survival protein Bcl-XL. Overall, this results in the suppression of T-cell or B-cell activation, proliferation, and cytokine secretion. PD-1 expression on T cells following chronic viral infection and on tumor-infiltrating lymphocytes has been shown to result in immune dysfunction characteristic of exhaustion, while blockade of PD-1 signaling has been shown to enhance T-cell proliferation and restore immune responses.

ANTIBODY A is a human Fc-silenced IgG1 bispecific antibody that can simultaneously bind to both TGFβR2 and PD-1. ANTIBODY A is designed to block the PD-1 axis and selectively targets TGFβ signaling blockade on activated PD-1-expressing T cells.

The amino acid sequences of the ANTIBODY A heavy and light chains are shown below. ANTIBODY A contains two different heavy chains, a TGFβR2 heavy chain (which binds TGFβR2) and a PD-1 heavy chain (which binds to PD-1), and a common light chain that pairs with each of the TGFβR2 and PD-1 heavy chains. Complementarity-determining regions (CDRs) 1, 2, and 3 (HCDRs specified according to Kabat (see Kabat et al., Sequences of Proteins of Immunological Interest, U.S. Dept. of Health and Human Services, Public Health Service, National Institutes of Health, 1991, (OCoLC) 1138727707) and LCDRs specified according to IMGT (see Giudicelli et al., IMGT/V-QUEST: IMGT standardized analysis of the immunoglobulin (IG) and T cell receptor (TR) nucleotide sequences. Cold Spring Harb Protoc 2011 (6): 695-715)) of the variable heavy (VH) domain and the variable light (VL) domain are shown below. An antibody consisting of the TGFβR2 heavy chain amino acid sequence set forth in SEQ ID NO:10, the PD-1 heavy chain amino acid sequence set forth in SEQ ID NO:5, and the common light chain amino acid sequence set forth in SEQ ID NO: 15 (one light chain paired with each of the heavy chains) is termed “ANTIBODY A.”

The heavy chain (HC) of the anti-PD-1 binding domain and anti-TGFβR2 binding domain of ANTIBODY A, as well as the common light chain amino acid sequence, are depicted in Table 1.

TABLE 1
ANTIBODY A Sequences

| Antibody | |
|---|---|
| Feature | Sequences |
| anti-PD-1 HC | QVQLVQSGSELKKPGASVKVSCKASGYTFTRFALHWVRQAPGQGLEWMGWIDPNTGTPTFA |
| | QGVTGRFVFSLDTSVTTAYLQISSLKAEDTAVYYCARSLGYCDSDICYPNWIEDNWGQGTL |
| | VTVSSASTKGPSVFPLAPSSKSTSGGTAALGCLVKDYFPEPVTVSWNSGALTSGVHTEPAV |
| | LOSSGLYSLSSVVTVPSSSLGTQTYICNVNHKPSNTKVDKRVEPKSCDKTHTCPPCPAPEL |
| | GRGPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTDPPSRE |
| | EMTKNQVSLTCEVKGFYPSDIAVEWESNGQPENNYKTTPPVLDSDGSFFLYSKLTVDKSRW |
| | QQGNVFSCSVMHEALHNHYTQKSLSLSPG (SEQ ID NO: 5) |
| anti-TGFBR2 | EVQLVESGGGLVQPGGSLRLSCAASGFTFDIYAMTWVRQAPGKGLEWVSVISGSGGTTYYA |
| HC | DSVKGRFTISRDNSKNTLYLQMNSLRAEDTAVYYCARRGQYRDIVGATDYWGQGTLVTVSS |
| | ASTKGPSVFPLAPSSKSTSGGTAALGCLVKDYFPEPVTVSWKSGALTSGVHTFPAVLQSSG |
| | LYSLSSVVTVPSSSLGTQTYICKVNHKPSNTKVDKRVEPKSCDKTHTCPPCPAPELGRGPS |
| | VFLFPPKPKDTLMISRTPEVTCVVVDVSHEDPEVKFNWYVDGVEVHNAKTKPREEQYNSTY |
| | RVVSVLTVLHQDWLNGKEYKCKVSNKALPAPIEKTISKAKGQPREPQVYTKPPSREEMTKN |
| | QVSLKCLVKGFYPSDIAVEWESNGQPENNYKTTPPVLDSDGSFFLYSKLTVDKSRWQQGNV |
| | FSCSVMHEALHNHYTQKSLSLSPG (SEQ ID NO: 10) |
| common LC | DIQMTQSPSSLSASVGDRVTITCRASQSISSYLNWYQQKPGKAPKLLIYAASSLQSGVPSR |
| | FSGSGSGTDFTLTISSLQPEDFATYYCQQSYSTPPTFGQGTKVEIKRTVAAPSVFIFPPSD |
| | EQLKSGTASVVCLLNNFYPREAKVQWKVDNALQSGNSQESVTEQDSKDSTYSLSSTLTLSK |
| | ADYEKHKVYACEVTHQGLSSPVTKSFNRGEC (SEQ ID NO: 15) |

The VH and VL, as well as the CDRs of each of the VH and VL, are depicted in Tables 2-3 for the anti-PD-1 binding domain and the anti-TGFβR2 binding domain of ANTIBODY A, respectively.

TABLE 2
Amino Acid Sequences of Anti-PD-1 Binding Domain of ANTIBODY A

| Antibody | |
|---|---|
| Feature | Sequences |
| anti-PD-1 HC | REALH (SEQ ID NO: 1) |
| CDR1 | |
| anti-PD-1 HC | WIDPNTGTPTFAQGVTG (SEQ ID NO: 2) |
| CDR2 | |
| anti-PD-1 HC | SLGYCDSDICYPNWIEDN (SEQ ID NO: 3) |
| CDR3 | |
| common LC | QSISSY (SEQ ID NO: 11) |
| CDR1 | |
| common LC | AAS |
| CDR2 | |
| common LC | QQSYSTPPT (SEQ ID NO: 13) |
| CDR3 | |
| anti-PD-1 VH | QVQLVQSGSELKKPGASVKVSCKASGYTFTREALHWVRQAPGQGLEWMGWIDPNTGTPTFA |
| | QGVTGRFVFSLDTSVTTAYLQISSLKAEDTAVYYCARSLGYCDSDICYPNWIFDNWGQGTL |
| | VTVSS (SEQ ID NO: 4) |
| common VL | DIQMTQSPSSLSASVGDRVTITCRASQSISSYLNWYQQKPGKAPKLLIYAASSLQSGVPSR |
| | FSGSGSGTDFTLTISSLQPEDFATYYCQQSYSTPPTFGQGTKVEIK (SEQ ID NO: 14) |

TABLE 3
Amino Acid Sequences of Anti-TGFβR2 Binding Domain of ANTIBODY A

| Antibody | |
|---|---|
| Feature | Sequences |
| anti-TGFβR2 | IYAMT (SEQ ID NO: 6) |
| HC CDR1 | |
| anti-TGFβR2 | VISGSGGTTYYADSVKG (SEQ ID NO: 7) |
| HC CDR2 | |
| anti-TGFβR2 | RGQYRDIVGATDY (SEQ ID NO: 8) |
| HC CDR3 | |
| common LC | QSISSY (SEQ ID NO: 11) |
| CDR1 | |
| common LC | AAS |
| CDR2 | |
| common LC | QQSYSTPPT (SEQ ID NO: 13) |
| CDR3 | |
| anti-TGFBR2 | EVQLVESGGGLVQPGGSLRLSCAASGFTFDIYAMTWVRQAPGKGLEWVSVISGSGGTTYYA |
| VH | DSVKGRFTISRDNSKNTLYLQMNSLRAEDTAVYYCARRGQYRDIVGATDYWGQGTLVTVSS |
| | (SEQ ID NO: 9) |
| common VL | DIQMTQSPSSLSASVGDRVTITCRASQSISSYLNWYQQKPGKAPKLLIYAASSLQSGVPSR |
| | FSGSGSGTDFTLTISSLQPEDFATYYCQQSYSTPPTFGQGTKVEIK (SEQ ID NO: 14) |

In some embodiments, the anti-human PD-1 binding domain of the bispecific antibody of the present disclosure comprises a heavy chain variable region comprising an HCDR1 comprising the amino acid sequence set forth in SEQ ID NO:1, an HCDR2 comprising the amino acid sequence set forth in SEQ ID NO:2, and an HCDR3 comprising the amino acid sequence set forth in SEQ ID NO:3. In some embodiments, the anti-human PD-1 binding domain of the bispecific antibody of the present disclosure comprises a heavy chain variable region comprising the amino acid sequence as set forth in SEQ ID NO:4, or having at least 80%, 85%, 90%, or 95% sequence identity thereto. In some embodiments, the anti-human PD-1 binding domain of the bispecific antibody of the present disclosure comprises a heavy chain variable region (1) comprising an HCDR1 comprising the amino acid sequence set forth in SEQ ID NO:1, an HCDR2 comprising the amino acid sequence set forth in SEQ ID NO:2, and an HCDR3 comprising the amino acid sequence set forth in SEQ ID NO: 3, and (2) comprising the amino acid sequence as set forth in SEQ ID NO:4, or having at least 80%, 85%, 90%, or 95% sequence identity thereto.

In some embodiments, the anti-human TGFβR2 binding domain of the bispecific antibody of the present disclosure comprises a heavy chain variable region comprising an HCDR1 comprising the amino acid sequence set forth in SEQ ID NO:6, an HCDR2 comprising the amino acid sequence set forth in SEQ ID NO:7, and an HCDR3 comprising the amino acid sequence set forth in SEQ ID NO:8. In some embodiments, the anti-human TGFβR2 binding domain of the bispecific antibody of the present disclosure comprises a heavy chain variable region comprising the amino acid sequence as set forth in SEQ ID NO: 9, or having at least 80%, 85%, 90%, or 95% sequence identity thereto. In some embodiments, the anti-human TGFβR2 binding domain of the bispecific antibody of the present disclosure comprises a heavy chain variable region (1) comprising an HCDR1 comprising the amino acid sequence set forth in SEQ ID NO:6, an HCDR2 comprising the amino acid sequence set forth in SEQ ID NO:7, and an HCDR3 comprising the amino acid sequence set forth in SEQ ID NO:8, and (2) comprising the amino acid sequence as set forth in SEQ ID NO:9, or having at least 80%, 85%, 90%, or 95% sequence identity thereto.

In some embodiments, the anti-human PD-1 binding domain of the bispecific antibody of the present disclosure comprises a heavy chain variable region comprising an HCDR1 comprising the amino acid sequence set forth in SEQ ID NO:1, an HCDR2 comprising the amino acid sequence set forth in SEQ ID NO:2, and an HCDR3 comprising the amino acid sequence set forth in SEQ ID NO:3, and the anti-human TGFβR2 binding domain of the bispecific antibody comprises a heavy chain variable region comprising an HCDR1 comprising the amino acid sequence set forth in SEQ ID NO:6, an HCDR2 comprising the amino acid sequence set forth in SEQ ID NO:7, and an HCDR3 comprising the amino acid sequence set forth in SEQ ID NO:8. In some embodiments, the anti-human PD-1 binding domain of the bispecific antibody of the present disclosure comprises a heavy chain variable region comprising the amino acid sequence as set forth in SEQ ID NO:4, or having at least 80%, 85%, 90%, or 95% sequence identity thereto, and the anti-human TGFβR2 binding domain of the bispecific antibody comprises a heavy chain variable region comprising the amino acid sequence as set forth in SEQ ID NO:9, or having at least 80%, 85%, 90%, or 95% sequence identity thereto. In some embodiments, the anti-human PD-1 binding domain of the bispecific antibody of the present disclosure comprises a heavy chain variable region (1) comprising an HCDR1 comprising the amino acid sequence set forth in SEQ ID NO:1, an HCDR2 comprising the amino acid sequence set forth in SEQ ID NO:2, and an HCDR3 comprising the amino acid sequence set forth in SEQ ID NO:3, and (2) comprising the amino acid sequence as set forth in SEQ ID NO:4, or having at least 80%, 85%, 90%, or 95% sequence identity thereto, and the anti-human TGFβR2 binding domain of the bispecific antibody comprises a heavy chain variable region (1) comprising an HCDR1 comprising the amino acid sequence set forth in SEQ ID NO:6, an HCDR2 comprising the amino acid sequence set forth in SEQ ID NO:7, and an HCDR3 comprising the amino acid sequence set forth in SEQ ID NO:8, and (2) comprising the amino acid sequence as set forth in SEQ ID NO:9, or having at least 80%, 85%, 90%, or 95% sequence identity thereto.

In some embodiments, the anti-human PD-1 binding domain of the bispecific antibody of the present disclosure comprises (1) a heavy chain variable region comprising an HCDR1 comprising the amino acid sequence set forth in SEQ ID NO:1, an HCDR2 comprising the amino acid sequence set forth in SEQ ID NO:2, and an HCDR3 comprising the amino acid sequence set forth in SEQ ID NO:3, and (2) a light chain variable region comprising an LCDR1 comprising the amino acid sequence set forth in SEQ ID NO:11, an LCDR2 comprising the amino acid sequence set forth in SEQ ID NO:12, and an LCDR3 comprising the amino acid sequence set forth in SEQ ID NO: 13. In some embodiments, the anti-human PD-1 binding domain of the bispecific antibody of the present disclosure comprises (1) a heavy chain variable region comprising the amino acid sequence as set forth in SEQ ID NO:4, or having at least 80%, 85%, 90%, or 95% sequence identity thereto, and (2) a light chain variable region comprising the amino acid sequence as set forth in SEQ ID NO: 14, or having at least 80%, 85%, 90%, or 95% sequence identity thereto. In some embodiments, the anti-human PD-1 binding domain of the bispecific antibody of the present disclosure comprises: a heavy chain variable region (1) comprising an HCDR1 comprising the amino acid sequence set forth in SEQ ID NO:1, an HCDR2 comprising the amino acid sequence set forth in SEQ ID NO:2, and an HCDR3 comprising the amino acid sequence set forth in SEQ ID NO:3, and (2) comprising the amino acid sequence as set forth in SEQ ID NO: 4, or having at least 80%, 85%, 90%, or 95% sequence identity thereto; and a light chain variable region (1) comprising an LCDR1 comprising the amino acid sequence set forth in SEQ ID NO: 11, an LCDR2 comprising the amino acid sequence set forth in SEQ ID NO:12, and an LCDR3 comprising the amino acid sequence set forth in SEQ ID NO: 13, and (2) comprising the amino acid sequence as set forth in SEQ ID NO:14, or having at least 80%, 85%, 90%, or 95% sequence identity thereto.

In some embodiments, the anti-human TGFβR2 binding domain of the bispecific antibody of the present disclosure comprises (1) a heavy chain variable region comprising an HCDR1 comprising the amino acid sequence set forth in SEQ ID NO:6, an HCDR2 comprising the amino acid sequence set forth in SEQ ID NO:7, and an HCDR3 comprising the amino acid sequence set forth in SEQ ID NO:8, and (2) a light chain variable region comprising an LCDR1 comprising the amino acid sequence set forth in SEQ ID NO:11, an LCDR2 comprising the amino acid sequence set forth in SEQ ID NO:12, and an LCDR3 comprising the amino acid sequence set forth in SEQ ID NO:13. In some embodiments, the anti-human TGFβR2 binding domain of the bispecific antibody of the present disclosure comprises (1) a heavy chain variable region comprising the amino acid sequence as set forth in SEQ ID NO:9, or having at least 80%, 85%, 90%, or 95% sequence identity thereto, and (2) a light chain variable region comprising the amino acid sequence as set forth in SEQ ID NO: 14, or having at least 80%, 85%, 90%, or 95% sequence identity thereto. In some embodiments, the anti-human TGFβR2 binding domain of the bispecific antibody of the present disclosure comprises: a heavy chain variable region (1) comprising an HCDR1 comprising the amino acid sequence set forth in SEQ ID NO:6, an HCDR2 comprising the amino acid sequence set forth in SEQ ID NO:7, and an HCDR3 comprising the amino acid sequence set forth in SEQ ID NO:8, and (2) comprising the amino acid sequence as set forth in SEQ ID NO:9, or having at least 80%, 85%, 90%, or 95% sequence identity thereto; and a light chain variable region (1) comprising an LCDR1 comprising the amino acid sequence set forth in SEQ ID NO:11, an LCDR2 comprising the amino acid sequence set forth in SEQ ID NO: 12, and an LCDR3 comprising the amino acid sequence set forth in SEQ ID NO:13, and (2) comprising the amino acid sequence as set forth in SEQ ID NO:14, or having at least 80%, 85%, 90%, or 95% sequence identity thereto.

In some embodiments, the anti-human PD-1 binding domain of the bispecific antibody of the present disclosure comprises (1) a heavy chain variable region comprising an HCDR1 comprising the amino acid sequence set forth in SEQ ID NO:1, an HCDR2 comprising the amino acid sequence set forth in SEQ ID NO:2, and an HCDR3 comprising the amino acid sequence set forth in SEQ ID NO:3, and (2) a light chain variable region comprising an LCDR1 comprising the amino acid sequence set forth in SEQ ID NO:11, an LCDR2 comprising the amino acid sequence set forth in SEQ ID NO:12, and an LCDR3 comprising the amino acid sequence set forth in SEQ ID NO:13, and the anti-human TGFβR2 binding domain of the bispecific antibody comprises (1) a heavy chain variable region comprising an HCDR1 comprising the amino acid sequence set forth in SEQ ID NO:6, an HCDR2 comprising the amino acid sequence set forth in SEQ ID NO:7, and an HCDR3 comprising the amino acid sequence set forth in SEQ ID NO:8, and (2) a light chain variable region comprising an LCDR1 comprising the amino acid sequence set forth in SEQ ID NO: 11, an LCDR2 comprising the amino acid sequence set forth in SEQ ID NO:12, and an LCDR3 comprising the amino acid sequence set forth in SEQ ID NO:13. In some embodiments, the anti-human PD-1 binding domain of the bispecific antibody of the present disclosure comprises (1) a heavy chain variable region comprising the amino acid sequence as set forth in SEQ ID NO:4, or having at least 80%, 85%, 90%, or 95% sequence identity thereto, and (2) a light chain variable region comprising the amino acid sequence as set forth in SEQ ID NO:14, or having at least 80%, 85%, 90%, or 95% sequence identity thereto, and the anti-human TGFβR2 binding domain of the bispecific antibody comprises (1) a heavy chain variable region comprising the amino acid sequence as set forth in SEQ ID NO:9, or having at least 80%, 85%, 90%, or 95% sequence identity thereto, and (2) a light chain variable region comprising the amino acid sequence as set forth in SEQ ID NO:14, or having at least 80%, 85%, 90%, or 95% sequence identity thereto. In some embodiments, the anti-human PD-1 binding domain of the bispecific antibody of the present disclosure comprises: a heavy chain variable region (1) comprising an HCDR1 comprising the amino acid sequence set forth in SEQ ID NO: 1, an HCDR2 comprising the amino acid sequence set forth in SEQ ID NO: 2, and an HCDR3 comprising the amino acid sequence set forth in SEQ ID NO:3, and (2) comprising the amino acid sequence as set forth in SEQ ID NO:4, or having at least 80%, 85%, 90%, or 95% sequence identity thereto; and a light chain variable region (1) comprising an LCDR1 comprising the amino acid sequence set forth in SEQ ID NO:11, an LCDR2 comprising the amino acid sequence set forth in SEQ ID NO:12, and an LCDR3 comprising the amino acid sequence set forth in SEQ ID NO:13, and (2) comprising the amino acid sequence as set forth in SEQ ID NO: 14, or having at least 80%, 85%, 90%, or 95% sequence identity thereto, and the anti-human TGFβR2 binding domain of the bispecific antibody comprises: a heavy chain variable region (1) comprising an HCDR1 comprising the amino acid sequence set forth in SEQ ID NO:6, an HCDR2 comprising the amino acid sequence set forth in SEQ ID NO:7, and an HCDR3 comprising the amino acid sequence set forth in SEQ ID NO: 8, and (2) comprising the amino acid sequence as set forth in SEQ ID NO:9, or having at least 80%, 85%, 90%, or 95% sequence identity thereto; and a light chain variable region (1) comprising an LCDR1 comprising the amino acid sequence set forth in SEQ ID NO:11, an LCDR2 comprising the amino acid sequence set forth in SEQ ID NO: 12, and an LCDR3 comprising the amino acid sequence set forth in SEQ ID NO:13, and (2) comprising the amino acid sequence as set forth in SEQ ID NO: 14, or having at least 80%, 85%, 90%, or 95% sequence identity thereto.

In some embodiments, the bispecific antibody includes a human heavy chain and light chain constant region. In some embodiments, the heavy chain constant region comprises a CH1 domain and a hinge region. In some embodiments, the heavy chain constant region comprises a CH2 domain. In some embodiments, the heavy chain constant region comprises a CH3 domain. In some embodiments, the heavy chain constant region comprises CH1, CH2 and CH3 domains. If the heavy chain constant region includes substitutions, such substitutions modify the properties of the antibody (e.g., increase or decrease one or more of: Fc receptor binding, antibody glycosylation, the number of cysteine residues, effector cell function, or complement function). In some embodiments, the antibody is an IgG antibody. In some embodiments, the antibody is selected from the group consisting of IgG1, IgG2, IgG3, and IgG4.

Antibodies, such as ANTIBODY A, can be made, for example, by preparing and expressing synthetic genes that encode the recited amino acid sequences or by mutating human germline genes to provide a gene that encodes the recited amino acid sequences. Moreover, this antibody and other bispecific antibodies can be obtained, e.g., using one or more of the following methods.

Humanized antibodies can be generated by replacing sequences of the Fv variable region that are not directly involved in antigen binding with equivalent sequences from human Fv variable regions. General methods for generating humanized antibodies are provided by Morrison, S. L., Science, 229:1202-1207 (1985), by Oi et al., BioTechniques, 4:214 (1986), and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target, as described above, from germline immunoglobulin genes, or from synthetic constructs. The recombinant DNA encoding the humanized antibody can then be cloned into an appropriate expression vector.

Human germline sequences, for example, are disclosed in Tomlinson, I. A. et al., J. Mol. Biol., 227:776-798 (1992); Cook, G. P. et al., Immunol. Today, 16:237-242 (1995); Chothia, D. et al., J. Mol. Bio. 227:799-817 (1992); and Tomlinson et al., EMBO J., 14:4628-4638 (1995). The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

Other methods for humanizing antibodies can also be used. For example, other methods can account for the three dimensional structure of the antibody, framework positions that are in three dimensional proximity to binding determinants, and immunogenic peptide sequences. See, e.g., WO 90/07861; U.S. Pat. Nos. 5,693,762; 5,693,761; 5,585,089; 5,530,101; and U.S. Pat. No. 6,407,213; Tempest et al. (1991) Biotechnology 9:266-271. Still another method is termed “humaneering” and is described, for example, in U.S. 2005-008625.

Antibodies disclosed herein can include a human Fc region, e.g., a wild-type Fc region or an Fc region that includes one or more alterations. Antibodies may also have mutations that stabilize the disulfide bond between the two heavy chains of an immunoglobulin, such as mutations in the hinge region of IgG4, as disclosed in the art (e.g., Angal et al. (1993) Mol. Immunol. 30:105-08). See also, e.g., U.S. 2005-0037000.

Provided herein are compositions comprising a mixture of a bispecific antibody and one or more acidic variants thereof, e.g., wherein the amount of acidic variant(s) is less than about 80%, 70%, 60%, 60%, 50%, 40%, 30%, 30%, 20%, 10%, 5% or 1%. Also provided are compositions comprising a bispecific antibody comprising at least one deamidation site, wherein the pH of the composition is from about 5.0 to about 6.5, such that, e.g., at least about 90% of the bispecific antibodies are not deamidated (i.e., less than about 10% of the antibodies are deamidated). In some embodiments, less than about 5%, 3%, 2% or 1% of the antibodies are deamidated. The pH may be from 5.0 to 6.0, such as 5.5 or 6.0. In some embodiments, the pH of the composition is 5.5, 5.6, 5.7, 5.8, 5.9, 6.0, 6.1, 6.2, 6.3, 6.4 or 6.5.

An “acidic variant” is a variant of a polypeptide of interest which is more acidic (e.g., as determined by cation exchange chromatography) than the polypeptide of interest. An example of an acidic variant is a deamidated variant.

A “deamidated” variant of a polypeptide molecule is a polypeptide wherein one or more asparagine residue(s) of the original polypeptide have been converted to aspartate, i.e., the neutral amide side chain has been converted to a residue with an overall acidic character.

The term “mixture” as used herein in reference to a composition comprising a bispecific antibody means the presence of both the desired bispecific antibody and one or more acidic variants thereof. The acidic variants may comprise predominantly deamidated bispecific antibody, with minor amounts of other acidic variant(s).

In some embodiments, the binding affinity (K_{D}), on-rate (K_{D} on) and/or off-rate (K_{D} off) of the antibody that was mutated to eliminate deamidation is similar to that of the wild-type antibody, e.g., having a difference of less than about 5 fold, 2 fold, 1 fold (100%), 50%, 30%, 20%, 10%, 5%, 3%, 2% or 1%.

Bispecific antibodies of the disclosure can be prepared as full length antibodies or low molecular weight forms thereof (e.g., F(ab′)₂ bispecific antibodies, sc(Fv)₂ bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the CH3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

### Methods of Producing Antibodies

Antibodies may be produced in, for example, bacterial or eukaryotic cells. Some antibodies can be produced in bacterial cells, e.g., E. coli cells. Antibodies can also be produced in eukaryotic cells such as transformed cell lines (e.g., CHO, 293E, COS). In addition, antibodies can be expressed in a yeast cell such as Pichia (see, e.g., Powers et al., J Immunol Methods. 251:123-35 (2001)), Hansenula, or Saccharomyces. To produce the antibody of interest, a polynucleotide encoding the antibody is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5α, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EFla promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

Antibodies for use in methods described herein can be produced in mammalian cells. Exemplary mammalian host cells for expressing an antibody include Chinese Hamster Ovary (CHO) cells (including dhfr⁻ CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), human embryonic kidney 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal. For example, the cell is a mammary epithelial cell.

In an exemplary system for antibody expression, a recombinant expression vector(s) encoding the antibody heavy chains and the antibody light chains of a bispecific antibody (e.g., ANTIBODY A) is introduced into dhfr CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly.

The antibodies of the present disclosure can be isolated from inside or outside (such as from the medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

### Antibody Pharmaceutical Compositions and Administration

A bispecific antibody described herein can be formulated as a pharmaceutical composition for administration to a subject, e.g., to treat a disorder described herein. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see, e.g., Berge, S. M., et al. (1977) J. Pharm. Sci. 66:1-19).

The bispecific antibody can be administered to a subject, e.g., a subject in need thereof, for example, a human subject, by intravenous injection or infusion (IV).

The bispecific antibody can be administered as a fixed dose, or in a mg/kg patient weight dose. The dose can also be chosen to reduce or avoid production of antibodies against the bispecific antibody. Dosage regimens are adjusted to provide the desired response, e.g., a therapeutic response or a combinatorial therapeutic effect. Generally, doses of the bispecific antibody can be used in order to provide a subject with the agent in bioavailable quantities.

For example, doses in the range of about 0.1 mg/kg to about 30 mg/kg can be administered. In some embodiments, a subject is administered the antibody at a dose of about 0.1 mg/kg to about 10 mg/kg (e.g., a dose of about 0.1 mg/kg, 0.5 mg/kg, 1 mg/kg, 1.5 mg/kg, 2 mg/kg, 3 mg/kg, 5 mg/kg, 6 mg/kg, 7.5 mg/kg, or about 10 mg/kg). In other embodiments, a subject is administered the antibody at a dose of about 1 mg/kg to about 3 mg/kg (e.g., a dose of about 1 mg/kg, 2 mg/kg, or 3 mg/kg). With respect to doses or dosages, the term “about” is intended to denote a range that is +10% of a recited dose, such that, for example, a dose of about 3 mg/kg will be between 2.7 mg/kg and 3.3 mg/kg patient weight.

Dosage unit form or “fixed dose” or “flat dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent. Single or multiple dosages may be given. Alternatively, or in addition, the antibody may be administered via continuous infusion. For example, flat doses in the range of about 20 mg to 2500 mg can be administered. In some embodiments, a subject is administered the antibody at a dose of about 20 mg, 50 mg, 100 mg, 150 mg, 200 mg, 250 mg, 300 mg, 350 mg, 400 mg, 450 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, or 2500 mg. In some embodiments, a subject is administered the antibody at a dose of about 100 mg, 300 mg, 900 mg, 1500 mg, or 2000 mg.

A bispecific antibody dose can be administered, e.g., at a periodic interval over a period of time (a course of treatment) sufficient to encompass at least 2 doses, 3 doses, 5 doses, 10 doses, or more, e.g., weekly, biweekly (every two weeks), every three weeks, every four weeks, monthly, e.g., for between about 1 to 12 weeks. Factors that may influence the dosage and timing required to effectively treat a subject, include, e.g., the severity of the disease or disorder, formulation, route of delivery, previous treatments, the general health and/or age of the subject, and other diseases present. Moreover, treatment of a subject with a therapeutically effective amount of a compound can include a single treatment or, preferably, can include a series of treatments.

An exemplary flat dose dosing regimen comprises intravenous administration of a bispecific antibody described herein (e.g., ANTIBODY A) at a dosage of about 100 mg once every two weeks.

An further exemplary flat dose dosing regimen comprises intravenous administration of a bispecific antibody described herein (e.g., ANTIBODY A) at a dosage of about 300 mg once every two weeks.

An further exemplary flat dose dosing regimen comprises intravenous administration of a bispecific antibody described herein (e.g., ANTIBODY A) at a dosage of about 900 mg once every two weeks.

An further exemplary flat dose dosing regimen comprises intravenous administration of a bispecific antibody described herein (e.g., ANTIBODY A) at a dosage of about 1500 mg once every two weeks.

An further exemplary flat dose dosing regimen comprises intravenous administration of a bispecific antibody described herein (e.g., ANTIBODY A) at a dosage of about 2000 mg once every two weeks.

An exemplary flat dose dosing regimen comprises intravenous administration of a bispecific antibody described herein (e.g., ANTIBODY A) at a dosage of about 100 mg once every four weeks.

An further exemplary flat dose dosing regimen comprises intravenous administration of a bispecific antibody described herein (e.g., ANTIBODY A) at a dosage of about 300 mg once every four weeks.

An further exemplary flat dose dosing regimen comprises intravenous administration of a bispecific antibody described herein (e.g., ANTIBODY A) at a dosage of about 900 mg once every four weeks.

An further exemplary flat dose dosing regimen comprises intravenous administration of a bispecific antibody described herein (e.g., ANTIBODY A) at a dosage of about 1500 mg once every four weeks.

An further exemplary flat dose dosing regimen comprises intravenous administration of a bispecific antibody described herein (e.g., ANTIBODY A) at a dosage of about 2000 mg once every four weeks.

A pharmaceutical composition may include a “therapeutically effective amount” of a bispecific antibody described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects.

### Anticancer Therapies and Administration

Provided herein are methods of treating a cancer in a subject (e.g., a human subject), comprising administering to the subject a) an effective amount of ANTIBODY A, and b) an effective amount of or more of the following anticancer therapies: bevacizumab, FOLFIRI, FOLFOX, and/or cetuximab. Exemplary starting doses for each anticancer therapy are shown below in Table 4. Also shown are dose adjustments for each agent that can be implemented during the course of treatment.

TABLE 41
Dose adjustments for combination agents

| Combination Agent | Starting Dose | | Dose Level -1 | | Dose Level -2 | | Dose Level -3 |
|---|---|---|---|---|---|---|---|
| Oxaliplatin** | 85 | mg/m² | 65 | mg/m² | 50 | mg/m² | Discontinue |
| Irinotecan | 180 | mg/m² | 150 | mg/m² | 120 | mg/m² | |
| 5-FU bolus | 400 | mg/m² | 300 | mg/m² | 200 | mg/m² | |
| 5-FU infusion | 2400 | mg/m² | 2000 | mg/m² | 1600 | mg/m² | |
| Leucovorin* | 400 | mg/m² | 300 | mg/m² | 200 | mg/m² | |
| Cetuximab | 500 | mg/m² | 400 | mg/m² | 300 | mg/m² | |

| | | | |
|---|---|---|---|
| Bevacizumab | 5 | mg/kg | Dose reductions of bevacizumab not recommended |

*Levoleucovorin should be administered at 50% of leucovorin dose. Dosing of leucovorin is per institutional standards and may either match the 5-FU bolus dose or remain at 400 mg/m².
**If 5-FU is discontinued for toxicity, oxaliplatin should also be discontinued

### Indications

A combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) can be used to treat non-small cell lung cancer (NSCLC). In some embodiments, the NSCLC has squamous or nonsquamous histology. In some embodiments, the NSCLC is advanced or metastatic. In some embodiments, the subject has experienced disease progression after prior treatment (e.g., prior anti-PD-(L)1 therapy and/or anti-CTLA4 therapy).

A combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) can be used to treat squamous cell carcinoma of the head and neck (SCCHN). In some embodiments, the SCCHN comprises a primary squamous tumor of the oral cavity, oropharynx, hypopharynx, or larynx. In some embodiments, the SCCHN comprises human papilloma virus (HPV)-positive SCCHN. In some embodiments, the SCCHN is advanced or metastatic. In some embodiments, the subject has experienced disease progression after prior treatment (e.g., prior anti-PD-(L)1 therapy and/or anti-CTLA4 therapy).

A combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) can be used to treat cervical squamous cell carcinoma and endocervical adenocarcinoma (CESC). In some embodiments, the CESC comprises human papilloma virus (HPV)-positive CESC. In some embodiments, the CESC is advanced or metastatic. In some embodiments, the subject has experienced disease progression after prior treatment (e.g., prior anti-PD-(L)1 therapy and/or anti-CTLA4 therapy).

A combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) can be used to treat ovarian cancer. In some embodiments, the ovarian cancer comprises ovarian epithelial carcinoma, fallopian tube carcinoma, primary peritoneal carcinoma, or carcinosarcoma. In some embodiments, the ovarian cancer is advanced or metastatic. In some embodiments, the subject has experienced disease progression after prior treatment (e.g., prior anti-PD-(L)1 therapy and/or anti-CTLA4 therapy).

A combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) can be used to treat breast cancer. In some embodiments, the breast cancer comprises a triple negative breast cancer. In some embodiments, the triple negative breast cancer comprises HER2-negative, ER-negative, and PgR-negative breast cancer. In some embodiments, the breast cancer is advanced or metastatic. In some embodiments, the subject has experienced disease progression after prior treatment (e.g., prior anti-PD-(L)1 therapy and/or anti-CTLA4 therapy).

A combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) can be used to treat bladder cancer. In some embodiments, the bladder cancer comprises urothelial carcinoma. In some embodiments, the bladder cancer is advanced or metastatic. In some embodiments, the subject has experienced disease progression after prior treatment (e.g., prior anti-PD-(L)1 therapy and/or anti-CTLA4 therapy).

A combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) can be used to treat renal cell carcinoma. In some embodiments, the renal cell carcinoma is advanced or metastatic. In some embodiments, the subject has experienced disease progression after prior treatment (e.g., prior anti-PD-(L)1 therapy and/or anti-CTLA4 therapy).

A combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) can be used to treat melanoma. In some embodiments, the melanoma is cutaneous malignant melanoma. In some embodiments, the melanoma is advanced or metastatic. In some embodiments, the subject has experienced disease progression after prior treatment (e.g., prior anti-PD-(L)1 therapy and/or anti-CTLA4 therapy).

A combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) can be used to treat gastric adenocarcinoma. In some embodiments, gastric adenocarcinoma is advanced or metastatic. In some embodiments, the subject has experienced disease progression after prior treatment (e.g., prior anti-PD-(L)1 therapy and/or anti-CTLA4 therapy).

A combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) can be used to treat esophageal cancer. In some embodiments, the esophageal cancer is advanced or metastatic. In some embodiments, the subject has experienced disease progression after prior treatment (e.g., prior anti-PD-(L)1 therapy and/or anti-CTLA4 therapy).

A combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) can be used to treat gastroesophageal adenocarcinoma. In some embodiments, the gastroesophageal adenocarcinoma is advanced or metastatic. In some embodiments, the subject has experienced disease progression after prior treatment (e.g., prior anti-PD-(L)1 therapy and/or anti-CTLA4 therapy).

A combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) can be used to treat malignant pleural mesothelioma. In some embodiments, the malignant pleural mesothelioma is advanced or metastatic. In some embodiments, the subject has experienced disease progression after prior treatment (e.g., prior anti-PD-(L)1 therapy and/or anti-CTLA4 therapy).

A combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) can be used to treat pancreatic adenocarcinoma. In some embodiments, the pancreatic adenocarcinoma is advanced or metastatic. In some embodiments, the subject has experienced disease progression after prior treatment (e.g., prior anti-PD-(L)1 therapy and/or anti-CTLA4 therapy).

A combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) can be used to treat colorectal cancer (CRC). In some embodiments, the CRC comprises microsatellite stable colorectal cancer (MSS-CRC). In some embodiments, the CRC comprises deficient mismatch repair (dMMR)/high microsatellite instability (MSI-H) colorectal cancer (dMMR/MSI-H CRC). In some embodiments, the CRC is advanced or metastatic. In some embodiments, the subject has experienced disease progression after prior treatment (e.g., prior anti-PD-(L)1 therapy; anti-CTLA4 therapy; fluoropyrimidine-, oxaliplatin-, and irinotecan-based chemotherapies; anti-VEGF and/or anti-EGFR therapies; trifluridine and tipiracil, regorafenib, and/or fruquintinib therapies). In some embodiments, the subject has experienced disease progression after later-line treatment such as with trifluridine/tipiracil hydrochloride (TAS-102) or regorafenib.

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) for use in the treatment of non-small cell lung cancer (NSCLC). In some embodiments, the NSCLC has squamous or nonsquamous histology. In some embodiments, the NSCLC is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) for use in the treatment of squamous cell carcinoma of the head and neck (SCCHN). In some embodiments, the SCCHN comprises a primary squamous tumor of the oral cavity, oropharynx, hypopharynx, or larynx. In some embodiments, the SCCHN comprises human papilloma virus (HPV)-positive SCCHN. In some embodiments, the SCCHN is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) for use in the treatment of cervical squamous cell carcinoma and endocervical adenocarcinoma (CESC). In some embodiments, the CESC comprises human papilloma virus (HPV)-positive CESC. In some embodiments, the CESC is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) for use in the treatment of ovarian cancer. In some embodiments, the ovarian cancer comprises ovarian epithelial carcinoma, fallopian tube carcinoma, primary peritoneal carcinoma, or carcinosarcoma. In some embodiments, the ovarian cancer is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) for use in the treatment of breast cancer. In some embodiments, the breast cancer comprises a triple negative breast cancer. In some embodiments, the triple negative breast cancer comprises HER2-negative, ER-negative, and PgR-negative breast cancer. In some embodiments, the breast cancer is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) for use in the treatment of bladder cancer. In some embodiments, the bladder cancer comprises urothelial carcinoma. In some embodiments, the bladder cancer is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) for use in the treatment of renal cell carcinoma. In some embodiments, the renal cell carcinoma is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) for use in the treatment of melanoma. In some embodiments, the melanoma is cutaneous malignant melanoma. In some embodiments, the melanoma is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) for use in the treatment of gastric adenocarcinoma. In some embodiments, gastric adenocarcinoma is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) for use in the treatment of esophageal cancer. In some embodiments, the esophageal cancer is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) for use in the treatment of gastroesophageal adenocarcinoma. In some embodiments, the gastroesophageal adenocarcinoma is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) for use in the treatment of malignant pleural mesothelioma. In some embodiments, the malignant pleural mesothelioma is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) for use in the treatment of pancreatic adenocarcinoma. In some embodiments, the pancreatic adenocarcinoma is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) for use in the treatment of colorectal cancer (CRC). In some embodiments, the CRC comprises microsatellite stable colorectal cancer (MSS-CRC). In some embodiments, the CRC comprises deficient mismatch repair (dMMR)/high microsatellite instability (MSI-H) colorectal cancer (dMMR/MSI-H CRC). In some embodiments, the CRC is advanced or metastatic. In some embodiments, the subject has experienced disease progression after prior treatment (e.g., prior anti-PD-(L)1 therapy; anti-CTLA4 therapy; fluoropyrimidine-, oxaliplatin-, and irinotecan-based chemotherapies; anti-VEGF and/or anti-EGFR therapies; trifluridine and tipiracil, regorafenib, and/or fruquintinib therapies). In some embodiments, the subject has experienced disease progression after later-line treatment such as with trifluridine/tipiracil hydrochloride (TAS-102) or regorafenib.

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) in the manufacture of a medicament for treating non-small cell lung cancer (NSCLC). In some embodiments, the NSCLC has squamous or nonsquamous histology. In some embodiments, the NSCLC is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) in the manufacture of a medicament for treating squamous cell carcinoma of the head and neck (SCCHN). In some embodiments, the SCCHN comprises a primary squamous tumor of the oral cavity, oropharynx, hypopharynx, or larynx. In some embodiments, the SCCHN comprises human papilloma virus (HPV)-positive SCCHN. In some embodiments, the SCCHN is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) in the manufacture of a medicament for treating cervical squamous cell carcinoma and endocervical adenocarcinoma (CESC). In some embodiments, the CESC comprises human papilloma virus (HPV)-positive CESC. In some embodiments, the CESC is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) in the manufacture of a medicament for treating ovarian cancer. In some embodiments, the ovarian cancer comprises ovarian epithelial carcinoma, fallopian tube carcinoma, primary peritoneal carcinoma, or carcinosarcoma. In some embodiments, the ovarian cancer is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) in the manufacture of a medicament for treating breast cancer. In some embodiments, the breast cancer comprises a triple negative breast cancer. In some embodiments, the triple negative breast cancer comprises HER2-negative, ER-negative, and PgR-negative breast cancer. In some embodiments, the breast cancer is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) in the manufacture of a medicament for treating bladder cancer. In some embodiments, the bladder cancer comprises urothelial carcinoma. In some embodiments, the bladder cancer is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) for use in the treatment of renal cell carcinoma. In some embodiments, the renal cell carcinoma is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) in the manufacture of a medicament for treating melanoma. In some embodiments, the melanoma is cutaneous malignant melanoma. In some embodiments, the melanoma is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) in the manufacture of a medicament for treating gastric adenocarcinoma. In some embodiments, gastric adenocarcinoma is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) in the manufacture of a medicament for treating esophageal cancer. In some embodiments, the esophageal cancer is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) in the manufacture of a medicament for treating gastroesophageal adenocarcinoma. In some embodiments, the gastroesophageal adenocarcinoma is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) in the manufacture of a medicament for treating malignant pleural mesothelioma. In some embodiments, the malignant pleural mesothelioma is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) in the manufacture of a medicament for treating pancreatic adenocarcinoma. In some embodiments, the pancreatic adenocarcinoma is advanced or metastatic. In some embodiments, the subject has experienced disease progression after prior treatment (e.g., prior anti-PD-(L)1 therapy and/or anti-CTLA4 therapy).

Another aspect comprises a combination treatment comprising one or more anticancer therapies (e.g., bevacizumab, FOLFIRI, FOLFOX, bevacizumab and FOLFIRI, bevacizumab and FOLFOX, or cetuximab) and a bispecific antibody described herein (e.g., ANTIBODY A) in the manufacture of a medicament for treating colorectal cancer (CRC). In some embodiments, the CRC comprises microsatellite stable colorectal cancer (MSS-CRC). In some embodiments, the CRC comprises deficient mismatch repair (dMMR)/high microsatellite instability (MSI-H) colorectal cancer (dMMR/MSI-H CRC). In some embodiments, the CRC is advanced or metastatic. In some embodiments, the subject has experienced disease progression after prior treatment (e.g., prior anti-PD-(L)1 therapy; anti-CTLA4 therapy; fluoropyrimidine-, oxaliplatin-, and irinotecan-based chemotherapies; anti-VEGF and/or anti-EGFR therapies; trifluridine and tipiracil, regorafenib, and/or fruquintinib therapies). In some embodiments, the subject has experienced disease progression after later-line treatment such as with trifluridine/tipiracil hydrochloride (TAS-102) or regorafenib.

The following are examples of the practice of the invention. They are not to be construed as limiting the scope of the invention in any way.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1: Combination Treatments of ANTIBODY A and Anticancer Therapies in Participants with Select Advanced or Metastatic Solid Tumors

This Example describes a Phase 1, first-in-human, multicenter, open-label, dose-escalation, and dose-expansion study to investigate the safety, tolerability, PK, pharmacodynamics, and preliminary clinical efficacy of ANTIBODY A in participants with select advanced or metastatic solid tumors.

Tumor types have been selected under the assumption that they have elevated TGFβ signature gene expression, based on bioinformatic data generated using publicly available datasets (TCGA; NCI 2022). Part 1a will consist of dose escalation using a statistical hybrid design to identify the maximum tolerated dose (MTD) and/or recommended dose for expansion(s) (RDE(s)) for further evaluation in Part 1b.

Part 1b will be a dose expansion to further characterize one or more RDEs in two cohorts; one cohort will encompass tumor types known to be sensitive to immune checkpoint inhibitors (ICIs), and the other cohort will focus on tumor types for which there are currently no ICIs indicated and/or those that are typically nonsensitive to ICIs.

ANTIBODY A monotherapy has shown preliminary signs of efficacy in participants with advanced/metastatic MSS-CRC in Part 1 of the present clinical trial. Based on this early signal and the ongoing unmet need for therapies in this patient population, the combination of ANTIBODY A with selected anticancer therapies will be explored in Part 2 of this study. In particular, combination with bevacizumab (Combination Group 1) was selected because of the demonstrated importance of anti-angiogenesis across the treatment paradigm for CRC, including in late lines; combination with chemotherapy (FOLFIRI+bevacizumab and FOLFOX+bevacizumab-Combination Groups 2 and 3, respectively) was selected because of the potential for chemotherapy to synergize with immunotherapy via, for example, induction of immunogenic cell death or elimination of immunosuppressive cells; and combination with cetuximab (Combination Group 4) was selected because of its role in antigen presentation and increase in immunotherapy efficacy in preclinical models.

### Inclusion Criteria

Participants are eligible to be included in the study only if all of the following criteria apply:
1. Ability to comprehend and willingness to sign a written informed consent form (ICF) for the study.
2. Aged 18 years or older inclusive at the time of signing the ICF.
3. Willing and able to conform to and comply with all Protocol requirements, including all scheduled visits and Protocol procedures.
4. Part 1a and 1b: Have experienced disease progression after treatment with, be intolerant to, or be ineligible for, or refused available therapies, including anti-PD-(L) 1 or anti-CTLA4 therapy if applicable, that are known to confer clinical benefit. For MSS-CRC in Part 1b, this must include participants who have received, are ineligible for, or refused prior treatment with fluoropyrimidine-, oxaliplatin-, and irinotecan-based chemotherapy for advanced/metastatic or adjuvant disease setting.
5. Note: Prior anti-PD-(L)1 therapy should not have been discontinued because of intolerance. Eastern Cooperative Oncology Group (ECOG) performance status score of 0 or 1.
6. Part 1b only: Willingness to undergo pre- and on-treatment tumor biopsy (core or excisional).
Part 2a and 2b, all Combination Groups: Willingness to undergo pre-treatment tumor biopsy (core or excisional).
Part 2a and 2b Combination Group 1 only: Willingness to undergo on-treatment tumor biopsy (core or excisional).
   Note:
      Archival formalin-fixed paraffin-embedded (FFPE) tissue is acceptable in lieu of a fresh pretreatment biopsy as long as the sample is ≤12 months old and collected after the last treatment line. Refer to the Laboratory Manual for details on sample adequacy.
      If fresh biopsy is not feasible and no archival tissue≤12 months old and collected after the last treatment line is available, other archival FFPE tissue may be acceptable in consultation with medical monitor/sponsor.
      Fresh tumor biopsies must be taken from nontarget lesions.
      The biopsied or resected lesion should not have been in the field of prior irradiation unless there is documented progression of the lesion.
      On-treatment biopsies should be collected from the same tumor lesion that was biopsied at baseline when possible.
7. Willingness to avoid pregnancy or fathering children based on the criteria below.
   a. Male participants with reproductive potential must agree to take appropriate precautions to avoid fathering children from screening through 180 days after the last dose of study drug and must refrain from donating sperm during this period. Permitted methods in preventing pregnancy should be communicated to the participants and their understanding confirmed.
   b. Female participants who are woman of childbearing potential (WOCBP) must have a negative serum pregnancy test at screening and a negative urine pregnancy test before the first dose on Day 1 and must agree to take appropriate precautions to avoid pregnancy from screening through 190 days after the last dose of study drug/treatment and must refrain from donating oocytes during this period. Permitted methods in preventing pregnancy should be communicated to the participants and their understanding confirmed.
   c. Female participants not considered to be of childbearing potential as defined in are eligible.
8. Part 1a only: Histologically or cytologically confirmed advanced or metastatic solid tumors as follows:
   Bladder Cancer (BC)
      Mixed histology with predominant urothelial carcinoma component is allowed.
      Pure small-cell carcinoma, pure adenocarcinoma, and pure squamous cell carcinoma are excluded.
   Cervical Squamous Cell Carcinoma and Endocervical Adenocarcinoma (CESC)
   Esophageal Cancer (ESCA)
   Gastric Adenocarcinoma (GC)
   Gastroesophageal Junction (GEJ)
   Cutaneous Malignant Melanoma (Mel)
   Malignant Pleural Mesothelioma (MPM)
   Non-small Cell Lung Cancer (NSCLC)
      Either squamous or nonsquamous histology is allowed. For mixed histology, there must be a predominant histology.
      Mixed small-cell and non-small cell lung cancer histology is excluded.
      Tumors should not exhibit mutations in EGFR, ALK, ROS1, or BRAF.
   Ovarian Cancer (OC)
      Ovarian epithelial carcinoma, fallopian tube carcinoma, primary peritoneal carcinoma, or carcinosarcoma.
      Sertoli-Leydig or germ cell cancers are excluded.
   Renal Cell Carcinoma (RCC)
   Squamous Cell Carcinoma of the Head and Neck (SCCHN)
      Histologically or cytologically confirmed squamous cell carcinoma of the oral cavity, oropharynx, hypopharynx, or larynx not amenable to local therapy with curative intent (surgery or radiation with or without chemotherapy). Carcinoma of the nasopharynx, salivary gland, or nonsquamous histologies are excluded.
      Both ICI-naive and ICI-exposed participants are eligible.
   Triple-Negative Breast Cancer (TNBC)
      HER2-negative, ER-negative, and PgR-negative breast cancer
   Pancreatic Adenocarcinoma (PAAD)
   Colorectal Cancer (CRC)
9. Part 1a only: Participants who have been previously treated with PD-1 inhibitor must undergo a washout period of 5 months or 5 times the half-life of the last PD-1, whichever is shorter, before the first dose of study drug.
10. Part 1b only: Histologically or cytologically confirmed advanced or metastatic solid tumors as follows (the sponsor may limit enrollment to certain tumor types. Enrollment in Cohort ICI-Sensitive will start with the initially selected tumor types of SCCHN and GEJ/gastric cancer):
   Cohort ICI-Sensitive
      BC
         Mixed histology with predominant urothelial carcinoma component is allowed.
         Pure small-cell carcinoma, pure adenocarcinoma, and pure squamous cell carcinoma are excluded.
      CESC
      ESCA
      GC
      GEJ
      Mel
      MPM
      NSCLC
         Either squamous or nonsquamous histology is allowed. For mixed histology, there must be a predominant histology.
         Mixed small-cell and non-small cell lung cancer histology is excluded.
         Tumors should not exhibit mutations in EGFR, ALK, ROS1, or BRAF.
      OC
         Ovarian epithelial carcinoma, fallopian tube carcinoma, primary peritoneal carcinoma, or carcinosarcoma.
         Sertoli-Leydig or germ cell cancers are excluded.
      RCC
      SCCHN
         Histologically or cytologically confirmed squamous cell carcinoma of the oral cavity, oropharynx, hypopharynx, or larynx not amenable to local therapy with curative intent (surgery or radiation with or without chemotherapy)
         Carcinoma of the nasopharynx, salivary gland, or nonsquamous histologies are excluded.
         Both ICI-naive and ICI-exposed participants are eligible.
      TNBC
         HER2-negative, ER-negative, and PgR-negative breast cancer
      dMMR/MSI-H CRC
   Cohort ICI-Nonsensitive
      PAAD
      MSS-CRC
11. Presence of measurable disease according to RECIST v1.1.
   Note:
      Tumor lesions situated in a previously irradiated area, or in an area subjected to other locoregional therapy, are not considered measurable unless there has been demonstrated progression in the lesion.
12. For Part 2a and Part 2b, the Combination Group-specific requirements are as follows:
   Combination Group 1 (ANTIBODY A in combination with bevacizumab):
      Diagnosis of advanced/metastatic MSS-CRC and
      Have received, are ineligible for, or refused prior treatment with fluoropyrimidine-, oxaliplatin-, and irinotecan-based chemotherapy for advanced/metastatic or neoadjuvant/adjuvant disease setting.
   Combination Group 2 (ANTIBODY A in combination with FOLFIRI+bevacizumab)
      Diagnosis of advanced/metastatic MSS-CRC and
      Have received, are ineligible for, or refused prior treatment with a fluoropyrimidine and oxaliplatin and have not received irinotecan and
      Are indicated for treatment with FOLFIRI+bevacizumab.
   Combination Group 3 (ANTIBODY A in combination with FOLFOX+bevacizumab)
      Diagnosis of advanced/metastatic MSS-CRC and
      No prior treatment for advanced/metastatic disease. If treated with neoadjuvant/adjuvant therapy, no recurrence within 12 months of therapy completion
      Indicated for treatment with FOLFOX+bevacizumab.
   Combination Group 4 (ANTIBODY A in combination with cetuximab):
      Diagnosis of advanced/metastatic MSS-CRC without an activating RAS (KRAS, NRAS, HRAS) or BRAF mutation or HER2 overexpression (by IHC, ISH, and/or NGS)
      Have received prior treatment with an EGFR inhibitor.

### Study Design

The study will be conducted in two separate parts: monotherapy and combination with other anticancer therapies.

Part 1a and Part 2a—Dose Escalation

Part 1a and Part 2a will consist of dose escalation using a statistical hybrid design to identify the MTD and/or RDE(s) based on the safety, tolerability, PK, pharmacodynamics, and preliminary clinical efficacy of ANTIBODY A in monotherapy and in combination with other anticancer therapies. Part 1a of the study will include up to approximately 53 dose-limiting toxicity (DLT)-evaluable participants with select advanced or metastatic solid tumors who have progressed on, are intolerant to, or are ineligible for standard of care therapies. Part 2a will include approximately 48 DLT-evaluable participants (approximately 12 participants per combination group) with MSS-CRC and treat with ANTIBODY A in combination with bevacizumab, FOLFIRI+bevacizumab, FOLFOX+bevacizumab, or cetuximab, respectively.

Part 1a enrollment will commence at dose level (DL) 1 (ANTIBODY A 100 mg IV; see Table 5 with the option to escalate or de-escalate based on defined dose-limiting toxicity criteria. Part 2a may run in parallel with Part 1a and 1b. In the event that the study parts run in parallel for the same patient population, participants will be centrally assigned by the sponsor to the respective parts or groups. To ensure safety of the combination treatment, the ANTIBODY A starting dose in Part 2a will be at a minimum 1 dose level below the highest tested dose of ANTIBODY A in Part 1a declared tolerable by the SRC. In Part 1a and 2a, cohorts of at least 3 DLT-evaluable participants will be initially enrolled.

At each dose level in Part 1a, an observation period of at least 24 hours after the first participant's first dose will occur before subsequent participants can begin study treatment. No more than 2 participants per dose level will begin study treatment within a 24-hour period.

TABLE 5
Planned Dose Levels of ANTIBODY A During Part 1a

| Dose Level | ANTIBODY A Dose | |
|---|---|---|
| 1 | 100 | mg |
| 2 | 300 | mg |
| 3 | 900 | mg |
| 4 | 1500 | mg |
| 5 | 2000 | mg |

Dose-limiting toxicities occurring up to and including Day 28 will guide dose escalation/de-escalation and determination of the RDE(s) and/or MTD. However, late-onset, immune-mediated toxicities through 30 days after the last dose of study drug will be considered when assessing safety; therefore, a lower RDE or MTD may subsequently be determined based on relevant toxicities that become evident after Day 28. While determination of the MTD will follow the definition of a DLT that occurs up to and including Day 28, the totality of the available data will be considered for RDE(s).

### Justification for Dose

Part 1a—Dose Escalation

The proposed safe starting dose (SSD) attempts to minimize the exposure of participants with late-stage cancer to subtherapeutic dose levels of ANTIBODY A while balancing the safety risk associated with the nonclinical pharmacologic and toxicological profiles. This dose was determined from all nonclinical data and is considered to provide an acceptable benefit-risk profile.

Part 1b—Dose Expansion

ANTIBODY A will be administered at the RDE(s) identified during Part 1a. The decision regarding which dose(s) to select as the RDE(s) is based on the totality of data incorporating safety, PK, pharmacodynamic data, and preliminary clinical efficacy data, as available, obtained during this study.

The sponsor may decide to expand the study to more than 1 RDE with the purpose of dose optimization, if necessary. If more than 1 RDE is selected, the following criteria must be met:
RDEs do not have overlapping PK exposure.
RDE_{low} should not be lower than the minimal dose identified that exhibits pharmacological activity.
RDE_{high} should not exceed the MTD.

If more than 1 RDE is used within a particular disease group, participants will be randomly assigned to the different RDEs. Based on emerging data, Sponsor may decide to restrict expansion to 1 RDE depending on tumor type. Multiple tumor types from the protocol-defined tumor types may be combined into the same expansion cohort, if justified based on known pathophysiological and clinical similarities (e.g., gastric/GEJ/esophageal cancer).

Justification for Combination with Selected Anticancer Therapies

ANTIBODY A has shown preliminary signs of efficacy in participants with advanced/metastatic MSS-CRC. Based on this early signal and the ongoing unmet need for therapies in this patient population, the combination of ANTIBODY A with selected anticancer therapies will be explored in this study.

Rationale for Combination with Bevacizumab in Participants with Colorectal Cancer

Bevacizumab (e.g., Avastin® or approved biosimilars) is approved for patients with metastatic colorectal cancer in combination with IV 5-FU based chemotherapy for first- or second-line treatment. More recent data has also shown an overall survival benefit when bevacizumab is combined with trifluridine and tipiracil in heavily pretreated CRC patients. The Sunlight trial included patients who had previously been treated with fluoropyrimidine-, oxaliplatin-, and irinotecan-based chemotherapy, an anti-VEGF biological therapy, and if RAS wild-type, an anti-EGFR therapy and compared the combination of bevacizumab with trifluridine and tipiracil to trifluridine and tipiracil alone. The median OS was 10.8 months in the bevacizumab-containing group and 7.5 months with trifluridine and tipiracil alone (HR 0.61) (Prager G W, Taieb J, Fakih M, et al. Trifluridine-Tipiracil and Bevacizumab in Refractory Metastatic Colorectal Cancer. NEJM 2023; 388:1657-1667). These data and historical data in combination with chemotherapy in earlier lines of therapy for CRC highlights the importance of anti-angiogenesis across the treatment paradigm for CRC and will be explored in combination with ANTIBODY A in this trial.

The dose of bevacizumab to be used in this study will be 5 mg/kg IV every 2 weeks. This dose is the labeled dose for combination with bolus-IFL and is also the dose studied in the Sunlight trial described above.

Rationale for Combination with FOLFIRI+Bevacizumab and FOLFOX+Bevacizumab

Several trials have shown positive data for the use of the FOLFOX or FOLFIRI chemotherapy regimens in first- and second-line treatment advanced/metastatic CRC regardless of mutational status. Randomized trials have not demonstrated superiority of initial therapy with FOLFOX versus FOLFIRI or the reverse, and both are considered acceptable first line chemotherapy regimens per NCCN guidelines. The addition of bevacizumab to either FOLFOX or FOLFIRI has demonstrated an improvement in overall survival in the first and second line (Hurwitz H, Fehrenbacher L, Novotny W, et al. Bevacizumab plus Irinotecan, Fluorouracil, and Leucovorin for Metastatic Colorectal Cancer. N Engl J Med 2004; 250:2235-2242 and Giantonio B J, Catalano P J, Meropol N J, et al. Bevacizumab in combination with oxaliplatin, fluorouracil, and leucovorin (FOLFOX4) for previously treated metastatic colorectal cancer: results from the Eastern Cooperative Oncology Group Study E3200. J Clin Oncol 2007; 25:1539-1544). If an oxaliplatin-based chemotherapy regimen was selected for initial therapy, it is commonly changed to an irinotecan-based chemotherapy for the second-line therapy and vice versa. Based on the frequency of use of FOLFIRI and FOLFOX-based chemotherapy regimens with bevacizumab, it is important to evaluate the safety and efficacy of these regimens in combination with ANTIBODY A in patients with advanced/metastatic CRC. PD-1 inhibitors have been combined with standard of care chemotherapy and are approved therapies in several solid tumors including NSCLC, mesothelioma, HNSCC, gastric cancer, esophageal cancer, cervical cancer, biliary tract cancer, TNBC, and endometrial cancer. Chemotherapy may influence an immune response to tumors via induction of immunogenic cell death, elimination of immunosuppressive cells, or sensitization of tumor cells to immune effector cells, which provides a rationale for their combination with immunotherapeutic interventions (Bracci L, Schiavoni G, Sistigu A, Belardelli F. Immune-based mechanisms of cytotoxic chemotherapy: implications for the design of novel and rationale-based combined treatments against cancer. Cell Death Differ 2014; 21:15-25 and Hato S V, Khong A, de Vries I J, Lesterhuis W J. Molecular pathways: the immunogenic effects of platinum-based chemotherapeutics. Clin Cancer Res 2014; 20:2831-2837). Preclinical models have shown that both irinotecan and oxaliplatin have a synergistic effect when combined with immune checkpoint inhibitors (Liu P, Chen J, Zhao L, et al. PD-1 blockage synergizes with oxaliplatin-based, but not cisplatin-based, chemotherapy of gastric cancer. Oncoimmunology 2022; 11: e2093518 and Iwai T, Sugimoto M, Wakita D, et al. Topoisomerase I inhibitor, irinotecan, depletes regulatory T cells and up-regulates MHC class I and PD-L1 expression, resulting in a supra-additive antitumor effect when combined with anti-PD-L1 antibodies. Oncotarget 2018; 9:31411-31421) and clinical studies have shown that irinotecan- and oxaliplatin-based chemotherapy can be safely and effectively combined with anti-PD(L)1 agents (Antoniotti C, Rossini D, Pietrantonio F, et al. Upfront FOLFOXIRI plus bevacizumab with or without atezolizumab in the treatment of patients with metastatic colorectal cancer (AtezoTRIBE): a multicentre, open-label, randomized, controlled, phase 2 trial. Lancet Oncol 2022; 23:876-887; Antoniotti C, Rossini D, Pietrantonio F, et al. Upfront Fluorouracil, Leucovorin, Oxaliplatin, and Irinotecan Plus Bevacizumab With or Without Atezolizumab for Patients With Metastatic Colorectal Cancer: Updated and Overall Survival Results of the ATEZOTRIBE Study. J Clin Oncol 2024; 42:2637-2644; and Lenz H J, Parikh A, Spigel D, et al. Modified FOLFOX6 plus bevacizumab with and without nivolumab for first-line treatment of metastatic colorectal cancer: phase 2 results from the CheckMate 9X8 randomized clinical trial. J Immunother Cancer 2024; 12: e008409).

### Rationale for Combination With Cetuximab

Patients with advanced/metastatic CRC considered RAS and RAF wild-type are commonly treated with anti-EGFR therapies. Approved agents include both panitumumab and cetuximab. Cetuximab is an EGFR antagonist and in preclinical models has been shown to lead to the promotion of antigen presentation and an increase in immunotherapy efficacy. Cetuximab is a IgG1 antibody and stimulates ADCC in addition to the downstream immune effects of EGFR binding which can lead to cytotoxic T cell recruitment and priming (Ferris R L, Lenz H J, Trotta A M, et al. Rationale for combination of therapeutic antibodies targeting tumor cells and immune checkpoint receptors: Harnessing innate and adaptive immunity through IgG1 isotype immune effector stimulation. Cancer Treat Rev 2018; 63:48-60 and Saoudi Gonzalez N, Ros J, Baraibar I, et al. Cetuximab as a Key Partner in Personalized Targeted Therapy for Metastatic Colorectal Cancer. Cancers 2024; 16:412). Panitumumab is not an IgG1 antibody and therefore is not expected to have the same level of synergy in combination with ANTIBODY A.

Cetuximab (Erbitux®) is recommended for use in patients with left-sided primary KRAS wild-type disease a) combined with chemotherapy in front-line therapy, b) combined with chemotherapy following progression on therapies not containing an EGFR inhibitor, and c) anti-EGFR rechallenge (NCCN guidelines NCCN 2024).

### Objectives and Endpoints

To evaluate the safety, tolerability, and dose-limiting toxicities (DLTs) and determine the MTD and/or RDE(s) of ANTIBODY A in participants with select advanced or metastatic solid tumors. The primary objective is evaluated by measuring (1) occurrence of DLTs, (2) incidence of treatment-emergent adverse events (TEAEs), assessed by physical examinations, evaluating changes in vital signs, left ventricular ejection fraction (LVEF), and electrocardiograms (ECGs), and through clinical laboratory blood and urine sample evaluations, and (3) incidence of TEAEs leading to study drug treatment interruptions and discontinuation of study drug.

The secondary objectives of the study are: (1) to determine the preliminary efficacy of ANTIBODY A in terms of objective response rate (ORR), disease control rate (DCR), and duration of response (DOR) in participants with select advanced malignancies or metastatic solid tumors; and (2) to evaluate the PK of ANTIBODY A in participants with select advanced malignancies or metastatic solid tumors. The secondary objectives are evaluated by measuring the following endpoints: (1) Objective response: complete response (CR) or partial response (PR), as determined by the investigator by radiographic disease assessment according to RECIST v1.1; Disease control: CR, PR, or stable disease (SD) as determined by the investigator by radiographic disease assessment according to RECIST v1.1, and DOR: time from earliest date of disease response (CR or PR) until earliest date of disease progression as determined by the investigator by radiographic disease assessment according to RECIST v1.1; and (2) PK parameters for ANTIBODY A, including Cₘₐₓ, tₘₐₓ, Cₘᵢₙ, AUC_{0-∞}, CL, V_{z}, and t_{1/2}, as deemed appropriate.

Exploratory objectives include: (1) to evaluate the pharmacodynamics of ANTIBODY A in participants with select advanced or metastatic solid tumors; (2) to explore biomarkers that predict pharmacologic activity and/or correlate with clinical safety or efficacy; (3) to assess the immunogenicity of ANTIBODY A and the immunogenicity of bevacizumab/cetuximab in combination (where applicable) in participants with select advanced or metastatic solid tumors; (4) to evaluate the relationship between PK and the target engagement of ANTIBODY A via receptor occupancy in participants with select advanced or metastatic solid tumors; (5) to explore the relationship between ANTIBODY A exposure and response in participants with select advanced or metastatic solid tumors; and (6) to explore the relationship between ANTIBODY A exposure and safety in participants with select advanced or metastatic solid tumors. The exploratory objectives are evaluated by measuring the following endpoints: (1) changes in T-lymphocyte activation and cytokines in the blood as well as intratumoral T-lymphocyte changes will be analyzed and may be correlated to PK; (2) tumor immunophenotype at baseline measured by intratumoral CD8+T-lymphocytes as well as alterations in cells, genes, proteins, and RNA in either blood or biopsies that are relevant to the drug targets and tumor sensitivity and/or resistance; (3) immunogenicity, defined as the occurrence of ADAs specific to the antibodies being tested; (4) target receptor occupancy in peripheral blood samples; (5) PK and exposure data will be assessed and correlated to tumor measurements; and (6) PK and exposure data will be assessed and correlated to incidence and severity of TEAEs and DLTs.

### Example 2: Combination Treatment of ANTIBODY A and Anticancer Therapies in Participants with Metastatic Microsatellite Stable Colorectal Cancer

This Example describes a randomized, double-blind, Phase 3 study of standard-of-care (SOC) chemotherapy and bevacizumab with or without ANTIBODY A in the first-line treatment of metastatic microsatellite stable colorectal cancer (MSS CRC). Participants are at least 18 years of age with histologically or cytologically confirmed metastatic CRC not amenable to curative resection and no prior systemic therapy for metastatic disease. Participants who previously received neoadjuvant and/or adjuvant chemotherapy may be enrolled if there was no recurrence of disease within 12 months of completion of therapy. Participants who are MSI-H/dMMR or have tumors with a BRAF V600E mutation will not be eligible to participate in the study.

Patients with metastatic CRC will be randomized 1:1 to receive either 900 mg of ANTIBODY A once every two weeks (Q2W) by intravenous (IV) administration or placebo Q2W IV along with the standard regimen of FOLFOX and bevacizumab as follows:
ANTIBODY A 900 mg Q2W IV (D1 of each 14-day cycle) with oxaliplatin 85 mg/m² Q2W IV (D1), leucovorin 400 mg/m² Q2W IV (D1), 5-FU 400 mg/m² IV bolus followed by 2400 mg/m² (starting on D1), and bevacizumab 5 mg/kg Q2W IV (D1).
Placebo Q2W IV (D1 of each 14-day cycle) with oxaliplatin 85 mg/m² Q2W IV (D1), leucovorin 400 mg/m² Q2W IV (D1), 5-FU 400 mg/m² IV bolus followed by 2400 mg/m² (starting on D1), and bevacizumab 5 mg/kg Q2W IV (D1).

ANTIBODY A or placebo will be administered for approximately 2 years (52 cycles) along with FOLFOX and bevacizumab. Participants who complete 52 cycles of ANTIBODY A/placebo may continue the SOC therapy until they meet any of the discontinuation criteria. Randomization will be stratified by tumor sidedness (left, right, transverse), PD-L1 expression (≥1% or <1% by TAP), and presence of liver metastases (yes or no). For the stratification category of tumor sidedness, participants with an unknown location of the primary tumor will be grouped with those with tumors considered transverse.

Anti-PD-(L)1 agents combined with other agents have been tested across different lines of therapy in MSS CRC. While some studies have demonstrated a numerical improvement in endpoints and/or potential benefits in subpopulations, no immunotherapy combination regimen has demonstrated statistically significant benefit in survival or response rates. Combination chemotherapy with bevacizumab or an anti-EGFR antibody remains the SOC in the initial treatment of metastatic MSS CRC, and there remains a significant unmet need for therapy with long-term benefit.

Participants with metastatic MSS CRC who had received SOC therapy (including fluoropyrimidine, irinotecan, and oxaliplatin unless refused or intolerant) were eligible for treatment with ANTIBODY A in Part 1a and 1b of the Phase 1 study described in Example 1. A total of 105 participants with CRC were treated at 3 different dose levels in these monotherapy expansion/dose-optimization parts of the first-in-human study. Seventy-five participants (71.4%) had liver metastasis at baseline, and 98 (93.3%) had received 2 or more prior systemic regimens. Preliminary data showed 16 partial responses (ORR of 15.2%), including responders among participants with active liver metastases at baseline (9/75; 12.0%). All responders were MSS.

In Part 2 of the study described in Example 1, a preliminary analysis of 50 patients was performed (20 patients treated with ANTIBODY A with bevacizumab, 6 patients treated with ANTIBODY A with FOLFIRI and bevacizumab, 18 patients treated with ANTIBODY A with FOLFOX and bevacizumab, and 6 patients treated with ANTIBODY A with cetuximab). All patients were treated with 900 mg Q2W IV of ANTIBODY A. All patients completed the DLT evaluation period, and no DLTs were observed. One patient discontinued oxaliplatin in the ANTIBODY A+FOLFOX+bevacizumab group. No patients discontinued any treatment component across the other three treatment groups due to TEAEs. Partial responses in evaluable patients were observed in: 4/20 patients treated with ANTIBODY A+bevacizumab; 3/6 patients treated with ANTIBODY A+FOLFIRI+bevacizumab, 7/13 patients treated with ANTIBODY A+FOLFOX+bevacizumab, and ⅙ patients treated with ANTIBODY A+cetuximab. ANTIBODY A at 900 mg Q2W IV exhibited a PK comparable between treatment groups and the monotherapy, with no observed anti-drug antibody impact on PK in these combinations. Further analysis of the data for patients treated with ANTIBODY A+bevacizumab revealed that one patient was non-evaluable due to voluntary withdrawal and in the evaluable patients 5/19 had partial responses. These results suggest ANTIBODY A is well-tolerated at 900 mg Q2W IV in MSS CRC SoC combinations without DLTs.

### Inclusion Criteria

Participants are eligible to be included in the study only if all the following criteria apply:
1. Ability to comprehend and willingness to sign a written ICF for the study.
2. Aged 18 years or older, inclusive, at the time of signing the ICF.
3. Histologically or cytologically confirmed metastatic colorectal adenocarcinoma (Stage IV per the American Joint Committee on Cancer, Cancer Staging Manual, 8th Edition) not amenable to curative resection.
4. No prior systemic treatment for unresectable or metastatic CRC. Participants who previously received neoadjuvant and/or adjuvant therapy are allowed to enroll if there was no recurrence of disease within 12 months of last systemic therapy administration.
5. Radiographically measurable disease (based on local site investigator/radiology evaluation) per RECIST v1.1 criteria. Target lesions situated in a previously irradiated area are considered measurable if progression has been demonstrated in such lesions.
6. Availability of results for MSI-H/dMMR status, KRAS mutation status, and BRAF V600E mutation status at the time of randomization (to be obtained from the participant's medical record).
7. ECOG performance status score of 0 or 1.
8. Baseline archival tumor specimen available or willingness to undergo a pretreatment tumor biopsy to obtain the specimen. Tumor specimen must be evaluable for PD-L1 by the central laboratory and must be a formalin-fixed, paraffin-embedded tumor block or unstained slides from biopsy or resection of the primary tumor or metastasis. Participants can have an additional specimen submitted if the PD-L1 results are found to be not evaluable by the central laboratory.
9. Adequate organ function including hematological (platelets, hemoglobin, and absolute neutrophil count), hepatic (ALT, AST, total bilirubin, and albumin), renal (calculated CrCl and proteinuria), coagulation (INR or PT and aPTT), and cardiac (TnT or TnI). All laboratory studies except coagulation testing must be performed and the results confirmed to be within eligibility parameters within 7 days (including C1D1) prior to the start of study treatment.
10. Willingness to avoid pregnancy or father children based on the criteria below.
   a. Male participants with reproductive potential must agree to take appropriate precautions to avoid fathering children, including refraining from donating sperm, from screening through 1 month after the last dose of ANTIBODY A or placebo or through 6 months after the last dose of 5-FU or oxaliplatin, whichever occurs later (or longer as appropriate based on country-specific requirements).
   b. Female participants who are WOCBP must have a negative serum pregnancy test at screening and a negative urine pregnancy test before the first dose on Day 1 and must agree to take appropriate precautions to avoid pregnancy and refrain from donating oocytes from screening through 1 month after the last dose of ANTIBODY A or placebo or through 6 months after the last dose of 5-FU or bevacizumab or through 9 months after the last dose of oxaliplatin, whichever occurs later (or longer as appropriate based on country-specific requirements).
   c. Female participants not considered to be of childbearing potential are eligible.

### Exclusion Criteria

Participants are excluded from the study if any of the following criteria apply:

### Cancer History

1. Known MSI-H/dMMR status per local standard of practice as obtained from historical data in the participant's medical record.
2. BRAF V600E mutation as obtained from historical data in the participant's medical record.
3. History of other malignancy within 2 years of study entry (with the exceptions of basal cell or squamous cell carcinoma of the skin, superficial bladder cancer, prostate intraepithelial neoplasm, carcinoma in situ of the cervix, ductal carcinoma in situ of the breast, or other noninvasive or indolent malignancy). Note: Participants with multiple primary CRC tumors are eligible.
4. Untreated and/or progressing CNS metastases (e.g., evidence of new or enlarging brain metastasis or new neurological symptoms attributable to brain or CNS metastases). Note: Participants with previously treated and clinically stable brain or CNS metastases are eligible if all of the following apply:
   No CNS progression has been demonstrated on 2 scans at least 4 weeks apart
   CNS metastasis treatment has been completed at least 2 weeks prior to screening CNS imaging
   Any neurologic symptoms have returned to baseline
   There is no evidence of new or enlarging CNS metastasis or leptomeningeal disease or clinically significant CNS edema or hemorrhage
   Corticosteroids have not been required for symptoms of CNS metastases or toxicities of CNS metastasis treatment for at least 7 days before the first dose of study treatment.
5. Tumor known to invade or encase a major blood vessel or any history of clinically significant bleeding from tumor lesions within 30 days before enrollment.

### Prior and Concomitant Therapy

6. Currently receiving investigational therapy or participated in a study of an investigational agent and received study therapy or used an investigational device within 4 weeks of randomization.
7. Treatment with an anti-PD-(L)1 or anti-CTLA-4 antibody, or any other antibody or drug specifically targeting T-cell costimulation or checkpoint pathways, for any indication within the past 3 years.
8. Toxicity from prior therapy that has not recovered to ≤Grade 1 or baseline (with the exceptions of anemia not requiring transfusion support, fatigue, and any grade of alopecia). Paresthesia and/or peripheral sensory neuropathy of Grade 2 or higher due to prior chemotherapy (eg, oxaliplatin) are exclusionary.
9. Concurrent anticancer therapy (e.g., chemotherapy, radiation therapy, surgery, ablation, immunotherapy, biologic therapy, investigational therapy, or tumor embolization) other than the therapies being tested in this study. Note: Adjuvant hormonal therapy is permitted.
10. Received thoracic radiation of >30 Gy within 6 months of the first dose of study treatment. Note: Participants must have recovered from all radiation-related toxicities to ≤Grade 1 and not require corticosteroids for radiation-related toxicities. A 1-week washout is required for prior radiation.

### Medical History

11. History of organ transplant, including allogeneic stem cell transplantation.
12. Active autoimmune disease that has required systemic treatment in the past 2 years (i.e., with use of disease-modifying agents, corticosteroids, or immunosuppressive drugs). Replacement therapy (e.g., thyroxine, insulin, or physiologic corticosteroid replacement therapy for adrenal or pituitary insufficiency) is allowed. Note: A history of inflammatory bowel disease at any time is exclusionary unless treated definitively (e.g., proctectomy for ulcerative colitis) with confirmed remission by endoscopy and no associated symptoms or indication for treatment within the past 2 years.
13. Significant concurrent and/or uncontrolled medical condition, including but not limited to the following:
   a. Hepatic
      Known history of drug-induced liver injury; alcoholic liver disease; nonalcoholic steatohepatitis; primary biliary cirrhosis; ongoing extrahepatic obstruction caused by stones, cirrhosis of the liver, or portal hypertension; or moderate or severe ascites requiring 2 paracenteses within 4 weeks prior to the start of study treatment.
   b. Uncontrolled endocrine disease
      Diabetes mellitus
      Thyroid gland dysfunction
      Adrenal gland insufficiency
   c. Pulmonary
      Evidence of interstitial lung disease or active, noninfectious pneumonitis
      History of drug-induced pneumonitis or interstitial lung disease
      Bronchial stent placement within 6 months prior to enrollment
      Lymphangitic carcinomatosis
      Pleural effusion compromising respiratory function
   d. Cardiovascular
      Participants with impaired cardiac function or clinically significant cardiac disease, including but not limited to the following:
         New York Heart Association Class III or IV cardiac disease, including pre-existing clinically significant ventricular arrhythmia, congestive heart failure, or cardiomyopathy within the past 6 months
         Unstable angina pectoris
         Pericardial effusion that causes symptoms and/or affects cardiac function
         Acute myocardial infarction, transient ischemic attack, or cerebrovascular accident≤6 months before the first dose of study treatment
         SBP>150 mm Hg and/or DBP>90 mm Hg at screening or on C1D1. In the event that blood pressure is elevated, the participant may enroll if the average SBP and DBP for 3 blood pressure measurements is nonexclusionary. Participants with exclusionary blood pressure during screening may be eligible if adequate blood pressure control is achieved with appropriate management. Participants with a history of hypertensive crisis and/or hypertensive encephalopathy are excluded.
         History of myocarditis
         Other clinically significant heart disease
         Symptomatic peripheral vascular disease within 6 months of study enrollment
         Known vasculitis, aneurysms, and other vascular malformations of clinical significance
         New onset of deep vein thrombosis or pulmonary embolism within the past 3 months
         History of new thromboembolism while receiving therapeutic anticoagulation
         Active bleeding disorder or other history of clinically significant bleeding episodes within 30 days of enrollment
         History of hemoptysis (>2.5 mL/half teaspoon of bright red blood per episode) within 6 weeks of the first dose of study treatment
   e. GI
      Any bowel obstruction or GI perforation within 60 days prior to CID1, unless due to the primary colorectal tumor and addressed definitively (e.g., colon resection or colonic diversion). Participants with colonic stents are excluded.
      Requirement for enteral or parenteral nutrition at study entry.
      Active gastroduodenal ulcers within the past 3 months, unless determined to be healed via endoscopy.
14. Chronic or current active infectious disease requiring systemic antibiotics, antifungal, or antiviral treatment within 7 days of the first dose of study treatment.
15. Major surgery within 28 days of the first dose of study treatment or not recovered adequately from toxicities and/or complications from surgical intervention.
16. Nonhealing wounds, ulcers, or bone fractures.
17. Uncontrolled active HBV or HCV infection defined as follows (testing must be performed to determine eligibility):
   a. Chronic HBV infection with HBV DNA (viral load)>500 IU/mL. Note 1: Participants with chronic HBV infection who are on anti-HBV therapy and have HBV DNA<500 IU/mL may enroll. Participants with cleared prior HBV infection, defined as HBsAg negative, HBsAb positive, HBcAb positive, and undetectable HBV DNA, are also eligible for the study. Note 2: For participants with chronic HBV, anti-HBV therapy must be initiated prior to the initiation of study treatment and must continue while on study treatment and through 12 months after the end of study treatment. HBV prophylaxis can be administered to participants with cleared HBV infection per investigator discretion.
   b. Uncontrolled active HCV is defined as a positive HCV antibody result (or known history of hepatitis C) and a quantitative HCV RNA (viral load) result greater than the lower limit of detection for the assay. Anti-HCV-positive participants with no available confirmatory negative HCV RNA test results will be excluded. Note: Participants with a history of hepatitis C infection who received and completed treatment for HCV that was intended to eradicate the virus may participate if HCV therapy was completed at least 12 weeks prior to screening, HCV RNA levels are undetectable at screening, and they do not have cirrhosis and/or advanced fibrosis (F3 or F4). Participants with spontaneous clearance of hepatitis C with negative HCV RNA levels at screening are also eligible.
18. HIV positive, unless all of the following criteria are met:
   a. CD4+ count ≥350 μL.
   b. Undetectable viral load.
   c. Received highly active antiretroviral therapy.

### Medications

19. Current use of chronic systemic corticosteroids (i.e., >10 mg/day of prednisone or equivalent). Exceptions:
   Physiologic corticosteroid replacement therapy at doses of >10 mg/day of prednisone or equivalent for adrenal or pituitary insufficiency and in the absence of active autoimmune disease is permitted.
   Participants with any condition that requires the use of inhaled corticosteroids (e.g., asthma or chronic obstructive pulmonary disease exacerbation) may participate.
   Participants using topical, ocular, intra-articular, or intranasal corticosteroids (with minimal systematic absorption) may participate.
   Brief courses of corticosteroids for prophylaxis (e.g., contrast dye allergy) or study treatment-related standard premedication are permitted.
20. Received a live vaccine within 28 days before the first dose of study treatment. Note: Examples of live vaccines include but are not limited to the following: measles, mumps, rubella, chickenpox/zoster, yellow fever, rabies, BCG, and typhoid. Seasonal influenza vaccines for injection are generally killed-virus vaccines and are allowed; however, intranasal influenza vaccines are live, attenuated vaccines and are not allowed.
21. Current use of prohibited medication, including antineoplastic system therapy or biological therapy not specified in this Study; investigational agents other than ANTIBODY A; palliative radiation therapy administered within 1 week of the first dose of the study drug or radiation therapy to the thorax at a dose >30 Gy within 6 months of the first dose of the study drug (surgery, ablation, or radiotherapy for the primary purpose of tumor control is not permitted during this study); systemic immunosuppression for active autoimmune disease using immunosuppressive drugs or corticosteroids (>10 mg/day of prednisone or equivalent) within 2 years of Day 1 of study treatment and throughout the treatment period with ANTIBODY A/placebo (with the exception of acute treatment for an AE); chronic use of systemic corticosteroids (>10 mg/day of prednisone or equivalent, except as permitted); live vaccines within 28 days before first administration of study drug, throughout the treatment period of the study, and for a duration of 90 days after the last dose of study treatment; initiation or modification of therapeutic anticoagulation within 3 months period to the first dose of study drug; major surgery within 28 days prior to enrollment; systemic antibiotics, antifungal, or antiviral therapy to treat an infection within 7 days before the first dose of study treatment; probiotic dietary supplements; herbal therapies or dietary supplements unless needed to treat a known vitamin or mineral deficiency or used for symptom management or treatment of a medical condition (daily multivitamins or equivalent preparations are permitted); and medications that are known to result in QT prolongation should be avoided while a participant is receiving oxaliplatin.
22. Initiation or modification of therapeutic anticoagulation within 3 months prior to first dose of study treatment. A time-limited course of prophylactic anticoagulation (e.g., during hospitalization or after surgery) is permitted within 3 months of the first dose of study treatment if the anticoagulation therapy is completed prior to the start of study treatment.
23. Known hypersensitivity or severe reaction to any component of the ANTIBODY A formulation or a history of severe allergic reaction and/or anaphylaxis to a chimeric or humanized antibody or fusion protein.
24. Known hypersensitivity or severe reaction to any formulation component of the FOLFOX regimen or bevacizumab.
25. Known complete DPD deficiency as reported in the participant's medical record. Local guidelines and regulations for DPD activity testing and dose adjustments should be applied in case of partial deficiency.

### Other Exclusions

26. Women who are pregnant (Note: in Japan, this includes women who may possibly be pregnant based on medical interview) or breastfeeding. Note: In Japan, women who are breastfeeding and wish to enroll must discontinue breastfeeding prior to receiving study treatment. They must also refrain from breastfeeding during the course of the study and for 180 days after the last dose of study treatment.
27. Any condition that would, in the investigator's judgment, interfere with full participation in the study, including administration of study treatment and attending required study visits; pose a significant risk to the participant; or interfere with interpretation of study data.
28. The following participants are excluded in France: vulnerable populations according to article L.1121-6 of the French Public Health Code and adults under legal protection, or who are unable to express their consent per article L.1121-8 of the French Public Health Code, not affiliated to a social security per article L.1121-8-1 of the French Public Health Code.

### Justification for Dose

The dose of ANTIBODY A to be used in this study is 900 mg Q2W IV. The selection of this dose was based on an integrated assessment of clinical and preclinical data. During the dose escalation in the study described in Example 1, 1 DLT was observed at the highest dose level explored, 1500 mg Q2W IV, and no DLTs were observed at lower dose levels. Based on the totality of the data incorporating safety, PK, pharmacodynamic, and preliminary efficacy data, the doses of 300 mg Q2W IV, 600 mg Q2W IV, and 900 mg Q2W IV were selected for expansion. Participants were randomly assigned to these doses within the selected disease groups for dose optimization in the Phase 1 Study.

There was no discernable difference in overall safety at these RDEs. ANTIBODY A consistently demonstrated an acceptable safety profile across RDEs, with similar incidence of any TEAE, as well as of serious or higher-grade TEAEs, regardless of suspected relationship to ANTIBODY A. Tolerability was also similar across dose levels, with a generally low rate of TEAEs leading to permanent drug discontinuation, dose delays, or infusion interruptions.

There was also no discernable difference in efficacy in participants with CRC among these dose levels. In participants with CRC enrolled in these 3 RDEs, efficacy measured as ORR or DCR appeared numerically similar across RDEs (300 mg Q2W IV, ORR=17.4%, DRR=32.6%; 600 mg Q2W IV, ORR=11.1%, DRR=27.8%; 900 mg Q2W IV, ORR=14.6%, DRR=24.4%). Notably, shrinkage of liver metastases was observed in responders at each dose level, indicating consistent antitumor activity. Preliminary data suggest that responders receiving 900 mg Q2W IV may have a longer DOR than those treated with 300 mg or 600 mg Q2W IV. In line with these clinical observations, no significant association was observed between ANTIBODY A exposure and either safety or efficacy in exposure-safety and exposure-efficacy analyses, respectively.

A key factor driving the selection of the 900 mg Q2W IV dose is the dose-dependent impact of immunogenicity on PK. Based on preliminary data available, in the Phase 1 study, ANTIBODY A was associated with a relatively high incidence of treatment-emergent antidrug antibodies (ADAs; 81.3% of participants). While no impact of ADAs on PK was observed in 88.6% of ADA-positive participants, 14.9%, 13.6%, and 3.6% of those treated with 300 mg Q2W IV, 600 mg Q2W IV, and 900 mg Q2W IV, respectively, had ADA-related impacts on PK, defined as a ≥3-fold reduction in trough concentrations or a decrease to levels below the lower limit of quantification (LLOQ) during subsequent infusions. In some of these participants, ADA-mediated reductions in drug exposure resulted in loss of PD-1 target engagement.

The impact of ADAs on PK exhibited a dose-dependent trend. Trough concentrations could not be maintained in 14.9% and 13.6% of the assessable ADA-positive participants receiving ANTIBODY A 300 mg and 600 mg Q2W IV, respectively, while most ADA-positive participants (96.4%) receiving 900 mg Q2W IV maintained trough concentrations, with only 2 (3.6%) out of 55 participants showing a ≥3-fold decrease. Among those with ADA-related PK impact, trough concentrations fell below the LLOQ in 8 of 10 and 2 of 3 participants at the 300 mg and 600 mg Q2W IV doses, respectively. In contrast, for the 2 participants whose PK were impacted by ADAs at the 900 mg Q2W IV dose, trough concentrations remained above the LLOQ, with ANTIBODY A concentrations at the latest timepoints approximately 5- to 6-fold higher than the in vitro 50% effective concentration (1.4 μg/mL). These findings indicated that ADAs had minimal impact on ANTIBODY A PK at the 900 mg Q2W IV dose compared with the 300 mg Q2W IV and 600 mg Q2W IV doses.

Although no clear exposure-efficacy relationship was identified, interpretation may be limited by the low overall response rate. Selection of the 900 mg Q2W IV dose may therefore mitigate ADA-related reductions in exposure and maintain consistent target engagement over time, which may be critical for achieving and sustaining long-term efficacy.

Another important rationale for selecting the 900 mg Q2W IV dose is the target engagement required to achieve maximum efficacy, as supported by preclinical efficacy studies in a mouse model. These studies demonstrated that maintaining trough concentrations above the MLR EC90 (13 μg/mL) is necessary for optimal efficacy. Simulation results showed that 900 mg Q2W IV provided ANTIBODY A trough concentrations above the MLR EC90 throughout the dose administration interval in 97% of participants. In contrast, only 8% and 78% of participants receiving 300 mg and 600 mg Q2W IV, respectively, achieved full coverage of the MLR EC90.

The PK of ANTIBODY A are not expected to be affected by coadministration with FOLFOX plus bevacizumab. Preliminary results are available for participants treated with ANTIBODY A 900 mg Q2W IV in combination with bevacizumab 5 mg/kg (n=20), ANTIBODY A 900 mg Q2W IV in combination with FOLFIRI and bevacizumab (n=6), ANTIBODY A 900 mg Q2W IV in combination with FOLFOX and bevacizumab (n=18), and ANTIBODY A 900 mg Q2W IV in combination with cetuximab (n=6). The PK of ANTIBODY A were comparable to those observed in participants treated with ANTIBODY A monotherapy. In addition, the available data indicated that coadministration of ANTIBODY A 900 mg Q2W IV with these combinations did not result in any ADA-related impact on ANTIBODY A PK. These findings support the use of 900 mg Q2W IV for combination treatment in this study.

The 900 mg Q2W IV regimen has demonstrated consistent safety and tolerability in monotherapy across advanced/metastatic tumor types, providing a robust foundation for its use in combination therapy. Suitability for combination therapy has been preliminarily confirmed in the Phase 1 study. ANTIBODY A 900 mg Q2W IV was able to be safely combined with FOLFOX plus bevacizumab with no overlapping toxicities observed, supporting further development in this setting. Considering the totality of the efficacy, safety, pharmacodynamic, ADA, and PK data in monotherapy and combination therapy as available, 900 mg Q2W IV was the dose of ANTIBODY A selected for this study.

### Objectives and Endpoints

The primary endpoint of the study is progression-free survival (PFS) as assessed by the investigator.

A primary objective of this study is to evaluate the efficacy of the combination of ANTIBODY A and SOC therapy versus placebo and SOC therapy, as evaluated by measuring PFS (defined as the time from the date of randomization to the date of the first documented progression as determined by the investigator per RECIST v1.1 or death due to any cause).

A key secondary objective of this study is to evaluate the efficacy of the combination of ANTIBODY A and SOC therapy versus placebo and SOC therapy in the overall population, as evaluated by overall survival (OS; defined as the time from the date of randomization to the date of death due to any cause).

A further secondary objective of this study is to evaluate the efficacy of the combination of ANTIBODY A and SOC therapy versus placebo and SOC therapy, as evaluated by objective response rate (ORR; defined as CR or PR as determined by the investigator per RECIST v1.1) and DOR (defined as the time from the earliest date of documented response until the earliest date of disease progression as determined by the investigator per RECIST v1.1 or death to any cause, whichever occurs first).

A further secondary objective of this study is to evaluate the safety and tolerability of the combination of ANTIBODY A and SOC therapy versus placebo and SOC therapy, as evaluated by TEAEs per CTCAE v6.0 and TEAEs leading to dose interruption or study drug discontinuation.

A further secondary objective of this study is to evaluate changes from baseline in health-related quality of life assessments, as evaluated by patient-reported outcomes assessed by changes from baseline using the EQ-5D-5L, EORTC QLQ-C30, and FSCI-9, with a higher score indicating a more preferred health status.

An exploratory objective of this study is to evaluate the PK of ANTIBODY A when administered with FOLFOX and bevacizumab. PK parameters, including Cₘₐₓ, Tₘₐₓ, Cₘᵢₙ, and AUC₀₋ₜ, will be summarized using a population PK modeling method, as deemed appropriate.

A further exploratory objective of this study will be to evaluate the efficacy of the combination of ANTIBODY A and SOC therapy versus placebo and SOC therapy after starting new anticancer therapy, as evaluated by PFS2 (defined as the time from randomization to subsequent disease progression after initiation of new anticancer therapy based on investigator assessment (after that used for PFS) or death due to any cause, whichever comes first).

A further exploratory objective of this study will be to evaluate the use of curative intent therapy with the combination of ANTIBODY A and SOC therapy versus placebo and SOC therapy, with curative therapy defined as participants who underwent curative intent surgery and/or ablation and/or ablative dose radiotherapy to treat all active sites of disease.

A further exploratory objective of this study will be to assess the immunogenicity of ANTIBODY A in combination therapy, with immunogenicity defined as the occurrence of ADAs specific to ANTIBODY A.

A further exploratory objective of this study will be to identify biomarkers that may predict clinical outcomes of ANTIBODY A, FOLFOX, and bevacizumab in CRC, including as assessed by the relationship between baseline blood or tissue biomarkers, including tissue PD-L1 expression and clinical safety or efficacy endpoints.

A further exploratory objective of this study will be to assess health economics data in participants with CRC, as assessed by resource utilization associated with unplanned medical encounters.

### Example 3: Combination Treatment of ANTIBODY A and Bevacizumab in Participants with Metastatic Microsatellite Stable Colorectal Cancer

This Example describes an open-label, multicenter, single-arm Phase 2 clinical study evaluating ANTIBODY A in combination with bevacizumab in participants with advanced/metastatic microsatellite stable colorectal cancer whose disease has progressed following standard systemic therapies.

Participants with MSS CRC who have progressed after standard of care therapy have limited options and low expected objective response rates. After progression on SOC regimens, the disease is typically refractory, and available later-line options such as TAS-102 or regorafenib generally provide limited tumor shrinkage and short disease control. For example, in the SUNLIGHT trial, TAS-102 alone rarely shrank tumors (ORR 1.2%) and outcomes remained poor (median PFS 2.4 months; OS 7.5 months). When bevacizumab was added, the benefit was mainly improved disease control and survival (ORR 6.1%; PFS 5.6 months; OS 10.8 months), showing that in this setting current therapies help some patients live longer but seldom produce meaningful response rates, leaving a clear need for more effective therapies. See Prager et al., Trifluridine-Tipiracil and Bevacizumab in Refractory Metastatic Colorectal Cancer; NEJM 2023; 388:1657-1667.

In Part 2 of the study described in Example 1, a preliminary analysis of 20 patients treated with ANTIBODY A with bevacizumab was performed. All patients were treated with 900 mg Q2W IV of ANTIBODY A. All patients completed the DLT evaluation period, and no DLTs were observed. No patients discontinued any treatment component due to TEAEs. In 19 efficacy-evaluable patients as of the data cut-off, 5 partial responses were observed (ORR 26.3%) and 2 additional responses (iRECIST ORR 36.8%) were observed using iRECIST criteria (iPR) in patients with initial unconfirmed progression (iUPD). Of the 19 efficacy-evaluable patients, 100% had received prior treatment with fluoropyrimidine, oxaliplatin, irinotecan, 89% received prior anti-VEGF therapy, 32% received prior targeted, novel, or other regimen, 26% received prior anti-EGFR therapy, and 21% received prior treatment with Lonsurf (trifluridine/tipiracil), regorafenib, and/or fruquintinib. 53% received 2 prior regimens, 21% received 3 prior regimens, 16% received 4 prior regimens, and 10% received 5 or more prior regimens. The high 36.8% ORR (by iRECIST criteria) in this difficult-to-treat cohort of patients shows that the combination therapy of ANTIBODY A with bevacizumab exhibits antitumor activity in MSS CRC.

The combination of ANTIBODY A and bevacizumab is expected to provide a clinically meaningful antitumor effect in heavily pretreated participants with metastatic MSS CRC, a population for whom currently available later-line therapies rarely produce meaningful objective responses. Based on the observed tolerability and objective response rate in the preliminary combination-treatment dataset, this Phase 2 study will further evaluate the efficacy and safety of treatment with 900 mg Q2W IV of ANTIBODY A plus 5 mg/kg Q2W bevacizumab using objective response rate per RECIST v1.1 as the primary endpoint, with duration of response, progression-free survival, overall survival, safety, and ORR by iRECIST criteria as secondary endpoints.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a disorder in a human subject in need thereof, wherein the disorder is selected from the group consisting of a non-small cell lung cancer (NSCLC), a squamous cell carcinoma of the head and neck (SCCHN), a cervical squamous cell carcinoma and endocervical adenocarcinoma (CESC), an ovarian cancer, a breast cancer, a bladder cancer, a renal cell carcinoma, a melanoma, a gastric adenocarcinoma, an esophageal cancer, a gastroesophageal adenocarcinoma, a malignant pleural mesothelioma, a pancreatic adenocarcinoma, and a colorectal cancer (CRC), wherein the method comprises administering to the human subject a combination treatment comprising (1) a therapeutically effective amount of an anticancer therapy and (2) a therapeutically effective amount of a bispecific antibody that binds to human programmed death-1 (PD-1) and human transforming growth factor β receptor 2 (TGFβR2),
wherein the anticancer therapy comprises one or more of bevacizumab, FOLFIRI, FOLFOX, or cetuximab,
wherein the bispecific antibody comprises:
an anti-human PD-1 binding domain comprising a PD-1 heavy chain variable region and a PD-1 light chain variable region, wherein the PD-1 heavy chain variable region comprises heavy chain CDR1 (HCDR1) comprising the amino acid sequence RFALH (SEQ ID NO:1), heavy chain CDR2 (HCDR2) comprising the amino acid sequence WIDPNTGTPTFAQGVTG (SEQ ID NO:2), and heavy chain CDR3 (HCDR3) comprising the amino acid sequence SLGYCDSDICYPNWIFDN (SEQ ID NO:3), and wherein the PD-1 light chain variable region comprises light chain CDR1 (LCDR1) comprising the amino acid sequence QSISSY (SEQ ID NO: 11), light chain CDR2 (LCDR2) comprising the amino acid sequence AAS, and light chain CDR3 (LCDR3) comprising the amino acid sequence QQSYSTPPT (SEQ ID NO:13); and
an anti-human TGFβR2 binding domain comprising a TGFβR2 heavy chain variable region and a TGFβR2 light chain variable region, wherein the TGFβR2 heavy chain variable region comprises HCDR1 comprising the amino acid sequence IYAMT (SEQ ID NO:6), HCDR2 comprising the amino acid sequence VISGSGGTTYYADSVKG (SEQ ID NO:7), and HCDR3 comprising the amino acid sequence RGQYRDIVGATDY (SEQ ID NO:8), and wherein the TGFβR2 light chain variable region comprises LCDR1 comprising the amino acid sequence QSISSY (SEQ ID NO:11), LCDR2 comprising the amino acid sequence AAS, and LCDR3 comprising the amino acid sequence QQSYSTPPT (SEQ ID NO:13).

2. The method of claim 1, wherein the PD-1 heavy chain variable region comprises the amino acid sequence QVQLVQSGSELKKPGASVKVSCKASGYTFTRFALHWVRQAPGQGLEWMGWIDPNTGT PTFAQGVTGRFVFSLDTSVTTAYLQISSLKAEDTAVYYCARSLGYCDSDICYPNWIFDN WGQGTLVTVSS (SEQ ID NO:4) and the TGFβR2 heavy chain variable region comprises the amino acid sequence
(SEQ ID NO: 9)
EVQLVESGGGLVQPGGSLRLSCAASGFTFDIYAMTWVRQAPGKGLEWVS
VISGSGGTTYYADSVKGRFTISRDNSKNTLYLQMNSLRAEDTAVYYCAR
RGQYRDIVGATDYWGQGTLVTVSS.

3. The method of claim 1, wherein the PD-1 light chain variable region and the TGFβR2 light chain variable region each comprise the amino acid sequence
(SEQ ID NO: 14)
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPPTF
GQGTKVEIK.

4. The method of claim 1, wherein the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO:5, the PD-1 light chain comprises the amino acid sequence set forth in SEQ ID NO:15, the TGFβR2 heavy chain comprises the amino acid sequence set forth in SEQ ID NO: 10, and the TGFβR2 light chain comprises the amino acid sequence set forth in SEQ ID NO:15.

5. The method of claim 1, wherein the anticancer therapy comprises:
(i) bevacizumab,
(ii) bevacizumab and FOLFIRI,
(iii) bevacizumab and FOLFOX, or
(iv) cetuximab.

6.-46. (canceled)

47. The method of claim 1, wherein the human subject has experienced disease progression after prior treatment.

48. The method of claim 47, wherein the prior treatment comprises anti-PD-(L)1 therapy and/or anti-CTLA4 therapy.

49. The method of claim 1, wherein the disorder is nonamenable to curative treatments or procedures.

50. The method of claim 1, wherein the bispecific antibody is administered intravenously.

51. The method of claim 1, wherein the bispecific antibody is administered:
(i) at a dose of about 100 mg, about 300 mg, about 900 mg, about 1500 mg, or about 2000 mg, and/or
(ii) intravenously.

52.-53. (canceled)

54. The method of claim 51, wherein the bispecific antibody is administered once every two weeks.

55. (canceled)

56. The method of claim 1, wherein the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO:5, the PD-1 light chain comprises the amino acid sequence set forth in SEQ ID NO:15, the TGFβR2 heavy chain comprises the amino acid sequence set forth in SEQ ID NO:10, and the TGFβR2 light chain comprises the amino acid sequence set forth in SEQ ID NO:15, and wherein the bispecific antibody is administered intravenously once every two weeks at a dose of about 100 mg, about 300 mg, about 900 mg, about 1500 mg, or about 2000 mg.

57. The method of claim 51, wherein the bispecific antibody is administered once every four weeks.

58. (canceled)

59. The method of claim 1, wherein the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO:5, the PD-1 light chain comprises the amino acid sequence set forth in SEQ ID NO:15, the TGFβR2 heavy chain comprises the amino acid sequence set forth in SEQ ID NO: 10, and the TGFβR2 light chain comprises the amino acid sequence set forth in SEQ ID NO:15, and wherein the bispecific antibody is administered intravenously once every four weeks at a dose of about 100 mg, about 300 mg, about 900 mg, about 1500 mg, or about 2000 mg.

60. The method of claim 56, wherein the bispecific antibody is administered intravenously once every two weeks at a dose of about 900 mg.

61. The method of claim 1, wherein the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO:5, the PD-1 light chain comprises the amino acid sequence set forth in SEQ ID NO:15, the TGFβR2 heavy chain comprises the amino acid sequence set forth in SEQ ID NO:10, and the TGFβR2 light chain comprises the amino acid sequence set forth in SEQ ID NO:15, and wherein the bispecific antibody is administered intravenously once every two weeks at a dose of about 900 mg.

62. A method of treating metastatic microsatellite stable colorectal cancer in a human subject in need thereof, wherein the method comprises administering to the human subject a combination treatment comprising (1) a therapeutically effective amount of FOLFOX, (2) a therapeutically effective amount of bevacizumab, and (3) a therapeutically effective amount of a bispecific antibody that binds to human programmed death-1 (PD-1) and human transforming growth factor β receptor 2 (TGFβR2);
wherein the bispecific antibody comprises:
an anti-human PD-1 binding domain comprising a PD-1 heavy chain variable region and a PD-1 light chain variable region, wherein the PD-1 heavy chain variable region comprises heavy chain CDR1 (HCDR1) comprising the amino acid sequence RFALH (SEQ ID NO:1), heavy chain CDR2 (HCDR2) comprising the amino acid sequence WIDPNTGTPTFAQGVTG (SEQ ID NO:2), and heavy chain CDR3 (HCDR3) comprising the amino acid sequence SLGYCDSDICYPNWIFDN (SEQ ID NO:3), and wherein the PD-1 light chain variable region comprises light chain CDR1 (LCDR1) comprising the amino acid sequence QSISSY (SEQ ID NO: 11), light chain CDR2 (LCDR2) comprising the amino acid sequence AAS, and light chain CDR3 (LCDR3) comprising the amino acid sequence QQSYSTPPT (SEQ ID NO:13); and
an anti-human TGFβR2 binding domain comprising a TGFβR2 heavy chain variable region and a TGFβR2 light chain variable region, wherein the TGFβR2 heavy chain variable region comprises HCDR1 comprising the amino acid sequence IYAMT (SEQ ID NO:6), HCDR2 comprising the amino acid sequence VISGSGGTTYYADSVKG (SEQ ID NO:7), and HCDR3 comprising the amino acid sequence RGQYRDIVGATDY (SEQ ID NO:8), and wherein the TGFβR2 light chain variable region comprises LCDR1 comprising the amino acid sequence QSISSY (SEQ ID NO:11), LCDR2 comprising the amino acid sequence AAS, and LCDR3 comprising the amino acid sequence QQSYSTPPT (SEQ ID NO:13).

63. The method of claim 62, wherein the PD-1 heavy chain variable region comprises the amino acid sequence QVQLVQSGSELKKPGASVKVSCKASGYTFTRFALHWVRQAPGQGLEWMGWIDPNTGT PTFAQGVTGRFVFSLDTSVTTAYLQISSLKAEDTAVYYCARSLGYCDSDICYPNWIFDN WGQGTLVTVSS (SEQ ID NO:4) and the TGFβR2 heavy chain variable region comprises the amino acid sequence
(SEQ ID NO: 9)
EVQLVESGGGLVQPGGSLRLSCAASGFTFDIYAMTWVRQAPGKGLEWVS
VISGSGGTTYYADSVKGRFTISRDNSKNTLYLQMNSLRAEDTAVYYCAR
RGQYRDIVGATDYWGQGTLVTVSS.

64. The method of claim 62, wherein the PD-1 light chain variable region and the TGFβR2 light chain variable region each comprise the amino acid sequence
(SEQ ID NO: 14)
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPPTF
GQGTKVEIK.

65. The method of claim 62, wherein the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO: 5, the PD-1 light chain comprises the amino acid sequence set forth in SEQ ID NO: 15, the TGFβR2 heavy chain comprises the amino acid sequence set forth in SEQ ID NO:10, and the TGFβR2 light chain comprises the amino acid sequence set forth in SEQ ID NO: 15.

66. (canceled)

67. The method of claim 62, wherein the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO: 5, the PD-1 light chain comprises the amino acid sequence set forth in SEQ ID NO: 15, the TGFβR2 heavy chain comprises the amino acid sequence set forth in SEQ ID NO:10, and the TGFβR2 light chain comprises the amino acid sequence set forth in SEQ ID NO: 15, and wherein the bispecific antibody is administered intravenously once every two weeks at a dose of about 900 mg.

68. A method of treating metastatic microsatellite stable colorectal cancer in a human subject in need thereof, wherein the method comprises administering to the human subject a combination treatment comprising (1) a therapeutically effective amount of bevacizumab, and (2) a therapeutically effective amount of a bispecific antibody that binds to human programmed death-1 (PD-1) and human transforming growth factor β receptor 2 (TGFβR2);
wherein the bispecific antibody comprises:
an anti-human PD-1 binding domain comprising a PD-1 heavy chain variable region and a PD-1 light chain variable region, wherein the PD-1 heavy chain variable region comprises heavy chain CDR1 (HCDR1) comprising the amino acid sequence RFALH (SEQ ID NO:1), heavy chain CDR2 (HCDR2) comprising the amino acid sequence WIDPNTGTPTFAQGVTG (SEQ ID NO:2), and heavy chain CDR3 (HCDR3) comprising the amino acid sequence SLGYCDSDICYPNWIFDN (SEQ ID NO:3), and wherein the PD-1 light chain variable region comprises light chain CDR1 (LCDR1) comprising the amino acid sequence QSISSY (SEQ ID NO: 11), light chain CDR2 (LCDR2) comprising the amino acid sequence AAS (SEQ ID NO: 12), and light chain CDR3 (LCDR3) comprising the amino acid sequence QQSYSTPPT (SEQ ID NO:13); and
an anti-human TGFβR2 binding domain comprising a TGFβR2 heavy chain variable region and a TGFβR2 light chain variable region, wherein the TGFβR2 heavy chain variable region comprises HCDR1 comprising the amino acid sequence IYAMT (SEQ ID NO:6), HCDR2 comprising the amino acid sequence VISGSGGTTYYADSVKG (SEQ ID NO:7), and HCDR3 comprising the amino acid sequence RGQYRDIVGATDY (SEQ ID NO:8), and wherein the TGFβR2 light chain variable region comprises LCDR1 comprising the amino acid sequence QSISSY (SEQ ID NO:11), LCDR2 comprising the amino acid sequence AAS (SEQ ID NO: 12), and LCDR3 comprising the amino acid sequence QQSYSTPPT (SEQ ID NO:13).

69. The method of claim 68, wherein the PD-1 heavy chain variable region comprises the amino acid sequence QVQLVQSGSELKKPGASVKVSCKASGYTFTRFALHWVRQAPGQGLEWMGWIDPNTGT PTFAQGVTGRFVFSLDTSVTTAYLQISSLKAEDTAVYYCARSLGYCDSDICYPNWIFDN WGQGTLVTVSS (SEQ ID NO:4) and the TGFβR2 heavy chain variable region comprises the amino acid sequence
(SEQ ID NO: 9)
EVQLVESGGGLVQPGGSLRLSCAASGFTFDIYAMTWVRQAPGKGLEWVS
VISGSGGTTYYADSVKGRFTISRDNSKNTLYLQMNSLRAEDTAVYYCAR
RGQYRDIVGATDYWGQGTLVTVSS.

70. The method of claim 68, wherein the PD-1 light chain variable region and the TGFβR2 light chain variable region each comprise the amino acid sequence
(SEQ ID NO: 14)
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPPTF
GQGTKVEIK.

71. The method of claim 68, wherein the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO: 5, the PD-1 light chain comprises the amino acid sequence set forth in SEQ ID NO: 15, the TGFβR2 heavy chain comprises the amino acid sequence set forth in SEQ ID NO:10, and the TGFβR2 light chain comprises the amino acid sequence set forth in SEQ ID NO: 15.

72. (canceled)

73. The method of claim 68, wherein the bispecific antibody comprises a PD-1 heavy chain, a PD-1 light chain, a TGFβR2 heavy chain, and a TGFβR2 light chain, wherein the PD-1 heavy chain comprises the amino acid sequence set forth in SEQ ID NO: 5, the PD-1 light chain comprises the amino acid sequence set forth in SEQ ID NO: 15, the TGFβR2 heavy chain comprises the amino acid sequence set forth in SEQ ID NO:10, and the TGFβR2 light chain comprises the amino acid sequence set forth in SEQ ID NO: 15, and wherein the bispecific antibody is administered intravenously once every two weeks at a dose of about 900 mg.

74. The method of claim 68, wherein the cancer has progressed after treatment with a standard systemic therapy.

75. The method of claim 68, wherein the subject previously received:
(i) fluoropyrimidine-, oxaliplatin-, and irinotecan-based chemotherapy;
(ii) anti-VEGF and/or anti-EGFR therapy; and/or
(iii) trifluridine and tipiracil, regorafenib, and/or fruquintinib therapy.

76.-77. (canceled)

78. The method of claim 68, wherein the method achieves an objective response rate of at least 30% in a plurality of treated subjects as assessed by RECIST v1.1.


SEQUENCE LISTING (15 sequences)

SEQ ID NO: 1 - protein, 5 aa, synthetic construct
RFALH

SEQ ID NO: 2 - protein, 17 aa, synthetic construct
WIDPNTGTPTFAQGVTG

SEQ ID NO: 3 - protein, 18 aa, synthetic construct
SLGYCDSDICYPNWIFDN

SEQ ID NO: 4 - protein, 127 aa, synthetic construct
QVQLVQSGSELKKPGASVKVSCKASGYTFTRFALHWVRQAPGQGLEWMGWIDPNTGTPTFAQGVTGRFVFSLDTSVTTAYLQISSLKAEDTAVYYCARSLGYCDSDICYPNWIFDNWGQGTLVTVSS

SEQ ID NO: 5 - protein, 456 aa, synthetic construct
QVQLVQSGSELKKPGASVKVSCKASGYTFTRFALHWVRQAPGQGLEWMGWIDPNTGTPTFAQGVTGRFVFSLDTSVTTAYLQISSLKAEDTAVYYCARSLGYCDSDICYPNWIFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 5 aa, synthetic construct
IYAMT

SEQ ID NO: 7 - protein, 17 aa, synthetic construct
VISGSGGTTYYADSVKG

SEQ ID NO: 8 - protein, 13 aa, synthetic construct
RGQYRDIVGATDY

SEQ ID NO: 9 - protein, 122 aa, synthetic construct
EVQLVESGGGLVQPGGSLRLSCAASGFTFDIYAMTWVRQAPGKGLEWVSVISGSGGTTYYADSVKGRFTISRDNSKNTLYLQMNSLRAEDTAVYYCARRGQYRDIVGATDYWGQGTLVTVSS

SEQ ID NO: 10 - protein, 451 aa, synthetic construct
EVQLVESGGGLVQPGGSLRLSCAASGFTFDIYAMTWVRQAPGKGLEWVSVISGSGGTTYYADSVKGRFTISRDNSKNTLYLQMNSLRAEDTAVYYCARRGQYRDIVGATDYWGQGTLVTVSSASTKGPSVFPLAPSSKSTSGGTAALGCLVKDYFPEPVTVSWKSGALTSGVHTFPAVLQSSGLYSLSSVVTVPSSSLGTQTYICKVNHKPSNTKVDKRVEPKSCDKTHTCPPCPAPELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 6 aa, synthetic construct
QSISSY

SEQ ID NO: 12 - skipped

SEQ ID NO: 13 - protein, 9 aa, synthetic construct
QQSYSTPPT

SEQ ID NO: 14 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 15 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC